(19) United States
(12) Patent Application Publication
(10) Pub. No.: US 2026/0176305 A1
(43) Pub. Date: Jun. 25, 2026

(54) Anionically Functionalized Polypeptides, And Methods Of Making Same And Uses Thereof

(71) Applicant: Cornell University, Ithaca, NY (US)

(72) Inventors: Christopher Akinleye Alabi, Ithaca, NY (US); Azmain Alamgir, Ithaca, NY (US); Matthew Delisa, Ithaca, NY (US)

(21) Appl. No.: 19/129,876

(22) PCT Filed: Nov. 14, 2023
(86) PCT No.: PCT/US2023/079718

§ 371 Date: May 14, 2025

Related U.S. Application Data
(60) Provisional application No. 63/425,204, filed on Nov. 14, 2022.

(51) Int. Cl. C07K 14/00; A61K 9/00; A61K 31/00; A61K 38/00; A61K 47/60; A61K 47/64

(52) U.S. Cl. CPC C07K 14/001; A61K 9/0019; A61K 31/00; A61K 38/00; A61K 47/60; A61K 47/64; A61K 2300/00; C07K 2317/76

(57) ABSTRACT

Anionically functionalized polypeptides, methods of making same, and uses thereof. Anionically functionalized polypeptides comprise one or more anionic group(s), where each anionic group is covalently bound to an anionically functionalized polypeptide sidechain or N-terminus, and one or more cleavable group(s). Anionically functionalized polypeptides can be made by reaction of a bioconjugation reagent comprising one or more anionic group(s) with a polypeptide, which may be a modified polypeptide. Anionically functionalized polypeptides can be used, in various examples, to intracellularly deliver a polypeptide, in cell transfection methods, or in treatment methods.

### CROSS-REFERENCE TO RELATED APPLICATIONS

This application claims the benefit of U.S. Provisional Patent Application No. 63/425,204, filed Nov. 14, 2022; the contents of the above-identified application are hereby fully incorporated herein by reference in their entirety.

### SEQUENCE LISTING

The instant application contains a Sequence Listing, which has been submitted in .xml format and is hereby incorporated by reference in its entirety. Said .xml copy was created on Nov. 14, 2023, is named 018617_01699_ST26.xml, and is 15,722 bytes in size.

### BACKGROUND OF THE DISCLOSURE

Proteins possess a remarkable ability to perform broadly intricate functions in biology. Because of their diverse functional roles, proteins have naturally been investigated as potential therapies to combat human disease. In contrast to small-molecule drugs that have long dominated the pharmacopeia, protein-based therapeutics can offer reduced toxicities, enhanced bioavailabilities, highly specific modes of biological activity, etc. The growth of protein therapies in the clinic has been remarkable; in 2022 alone, nearly half of all FDA-approved drugs consisted of protein biologics such as, for example, monoclonal antibodies, cytokines, and hormones. It is of note, however, that almost all approved protein therapies operate in extracellular environments, which is largely due to the inability of proteins to spontaneously enter cells. Indeed, cells have undergone billions of years of evolution to prevent the unassisted passage of such large molecular weight, hydrophilic macromolecules through their hydrophobic plasma membranes.

The potential of proteins as intracellular therapeutic agents has been underscored by the use of novel protein scaffolds that target the “undruggable” proteome, as well as the recent application of gene-editing proteins to treat human disease. Translating these promising technologies for clinically relevant applications requires the development of efficacious delivery methods capable of transporting proteins into the cytosol of a cell. While DNA or RNA encoding for protein products can be introduced into cells via viral vectors or nanoparticles, these methods suffer from lack of temporal control, high immunogenicity, risk of genome integration, and unintended off-target effects in vivo. Viewed from this perspective, direct delivery of protein therapies into cells is a unique approach that overcomes some of the limitations and concerns associated with existing nucleic acid-based methods for treating different pathological conditions.

Over the years, several techniques have been pioneered for delivering proteins into the cytosol of cells, such as, for example, membrane disruption methods, chemical conjugation schemes (cell penetrating-peptides and hydrophobic “masking” compounds), and carrier-mediated approaches (polymeric assemblies, virus-like particles, and inorganic nanostructures). These strategies demonstrate varying degrees of success for in vitro delivery but suffer from key barriers that prevent their translation for clinical applications, including but not limited to low delivery efficiencies and instability in serum. The absence of FDA-approved protein delivery strategies highlights the considerable challenges associated with achieving successful intracellular delivery of protein therapies in vivo.

An emerging alternative strategy for protein delivery involves adapting methods that have already proven successful for the delivery of other biological drugs. In this vein, cationic lipids, best known for their ability to deliver nucleic acid cargos, have been recently explored as a promising platform for delivering proteins. Such cationic lipid carriers have enabled successful delivery of enzymes, CRISPR-Cas complexes, and inhibitory protein scaffolds with the potential for therapeutic use. However, the majority of these previous efforts require protein cargos to be genetically fused with anionic polypeptides or protein domains to promote electrostatic interactions with cationic lipids. Thus, the implementation of such strategies involves genetic manipulation to reengineer protein cargos that incorporate these anionic tags, which can be time consuming, may not always be tolerated by the cargo protein, and limits off-the-shelf proteins from being directly functionalized for delivery.

Recent advances in protein delivery approaches have also investigated charge-based complexation of proteins with cationic lipids. The majority of these strategies, however, rely on genetically encoding anionic polypeptides into the backbones or protein cargos or using naturally anionic protein complexes to facilitate complexation with lipid reagents.

### SUMMARY OF THE DISCLOSURE

The present disclosure provides, inter alia, anionically functionalized polypeptides, compositions, methods of treating, bioconjugation reagents, and methods of making anionically functionalized polypeptides.

In various examples, an anionically functionalized polypeptide comprises (or consists of) the following structure: D-G-X₁-L-(R)ₓ or a structural analog thereof or a pharmaceutically acceptable salt, a salt, a partial salt, a solvate, a polymorph, a prodrug, or the like thereof, or a stereoisomer or a mixture of stereoisomers, an isotopic variant, a tautomer, or the like thereof, where D comprises (or consists of) a peptide group; G comprises (or consists of) a conjugated group; X₁ comprises (or consists of) a cleavable group; L comprises (or consists of) a linking group; R comprises (or consists of) an anionic group; and x is 1, 2, 3, 4, 5, or 6. In various examples, the polypeptide comprises (or consists of) the following structure:

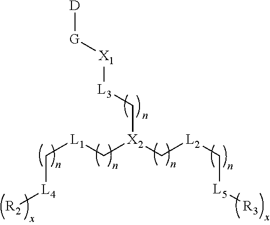

or a structural analog thereof or a pharmaceutically acceptable salt, a salt, a partial salt, a solvate, a polymorph, a prodrug, or the like thereof, or a stereoisomer or a mixture of stereoisomers, an isotopic variant, a tautomer, or the like thereof, where X₂ is chosen from an N group, a CH group, a P group, and a P═O group, or a structural analog thereof, L₁ and L₂ are independently optional and chosen from —OC(O)—, —C(O)O—, —NHC(O)—, —C(O)NH—, —OC(O)NH—, —NHC(O)O—,

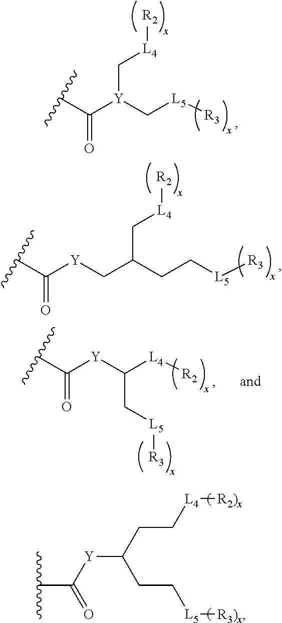

and structural analogs thereof, where Y is independently chosen from a NH group, an O group, an S group, a CH₂ group, an H group, and a CH group, and the like; L₃ is chosen from —OC(O)—, —C(O)O—, —NHC(O)—, —C(O)NH—, —OC(O)NH—, —NHC(O)O—, and structural analogs thereof; L₄ and L₅ are independently optional and chosen from alkyl groups and alkenyl groups, and the like; R₂ and R₃ are independently optional, wherein the anionically functionalized polypeptide comprises at least one R₂ and/or R₃, and chosen from —CO₂⁻, —CO₂H, —SO₃⁻, —SO₃H, —SO₂⁻, —PO₂H²⁻, —PO₃H⁻, and —PO₄H²⁻, polymeric groups comprising one or more of the anionic groups(s), and the like; n is independently at each occurrence 0, 1, 2, 3, 4, 5, or 6; and x is independently at each occurrence 1, 2, 3, 4, 5, or 6. In various examples, the polypeptide comprises (or consists of) the following structure:

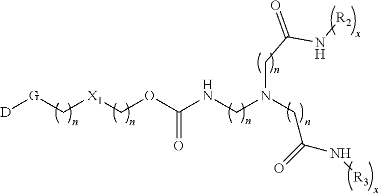

or a structural analog thereof or a pharmaceutically acceptable salt, a salt, a partial salt, a solvate, a polymorph, a prodrug, or the like thereof, or a stereoisomer or a mixture of stereoisomers, an isotopic variant, a tautomer, or the like thereof. In various examples, the polypeptide comprises (or consists of) the following structure:

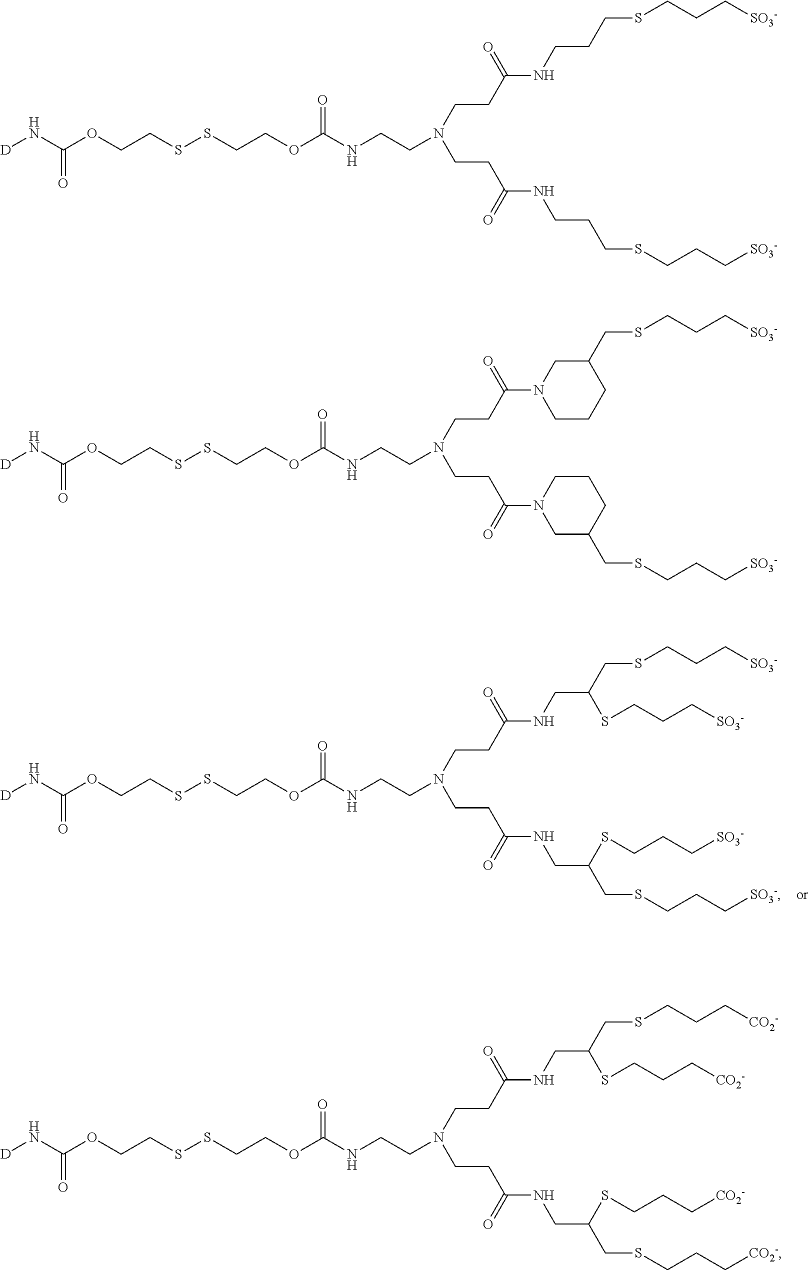

or a structural analog thereof or a pharmaceutically acceptable salt, a salt, a partial salt, a solvate, a polymorph, a prodrug, or the like thereof, or a stereoisomer or a mixture of stereoisomers, an isotopic variant, a tautomer, or the like thereof. In various examples, the peptide group is chosen from enzyme groups, receptor ligand groups, transcriptional factors, growth factor groups, antibody groups, peptide or protein immunogen groups, protein-based therapeutic agent groups, toxin groups, cytokine groups, hormone groups, fluorescent protein groups, any fragments thereof, any structural analogs thereof (such as, for example, modified structural analogs thereof), and the like, and any combination thereof. In various examples, the enzyme groups are chosen from Clustered Regularly Interspaced Short Palindromic Repeats (CRISPR) enzyme groups or the like or formed from an CRISPR enzyme or the like. In various examples, the conjugated group comprises (or consists of) the following structure:

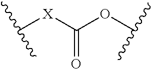

or a structural analog thereof, where X is chosen from an N group, an S group, and an O group, or the like. In various examples, the cleavable group is chosen from stimuli-cleavable bonds, light-cleavable bonds, ROS-cleavable bonds, and pH-cleavable bonds, and the like. In various examples, the stimuli-cleavable bond comprises (or consists of) a disulfide bond (such as, for example, redox-cleavable disulfide bond or the like).

In various examples, a composition comprising one or more of the anionically functionalized polypeptide(s) (e.g., anionically functionalized polypeptide(s) of the present disclosure). In various examples, the composition further comprises one or more cationic lipid(s) or the like or a combination of lipids or the like. In various examples, the composition comprises a plurality of lipoplexes or a plurality of lipid nanoparticles, or the like, where the anionically functionalized polypeptide(s) is/are independently disposed in or partially or completely encapsulated, or the like, by a lipid nanoparticle. In various examples, the composition is a pharmaceutical composition, and the composition further comprises one or more pharmaceutically acceptable excipient(s) or the like.

In various examples, a method of treating a subject with one or more symptoms(s), the method comprising: administering to a subject an effective amount of one or more of the anionically functionalized polypeptide(s) (e.g., anionically functionalized polypeptide(s) of the present disclosure), where at least one of the one or more symptom(s) of the subject is at least partially or completely alleviated.

In various examples, a bioconjugation reagent comprises (or consists of) the following structure: G′-X₁-L-(R)ₓ or a structural analog thereof or a pharmaceutically acceptable salt, a salt, a partial salt, a solvate, a polymorph, a prodrug, or the like thereof, or a stereoisomer or a mixture of stereoisomers, an isotopic variant, a tautomer, or the like thereof, where G′ comprises (or consists of) a conjugation group; X₁ comprises (or consists of) a cleavable group; L comprises (or consists of) a linking group; R comprises (or consists of) an anionic group; and x is 1, 2, 3, 4, 5, or 6. In various examples, the bioconjugation reagent comprises (or consists of) the following structure:

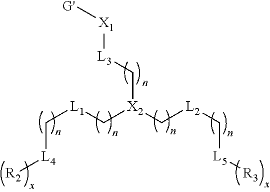

or a structural analog thereof or a pharmaceutically acceptable salt, a salt, a partial salt, a solvate, a polymorph, a prodrug, or the like thereof, or a stereoisomer or a mixture of stereoisomers, an isotopic variant, a tautomer, or the like thereof, where X₂ is chosen from an N group, a CH group, a P group, and a P═O group or a structural analog thereof, L₁ and L₂ are independently chosen from —OC(O)—, —C(O)O—, —NHC(O)—, —C(O)NH—, —OC(O)NH—, —NHC(O)O—,

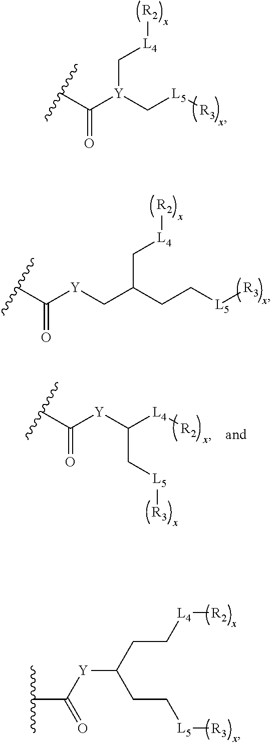

and structural analogs thereof, where Y is independently chosen from an NH group, an O group, an S group, a CH₂ group, an H group, and a CH group, and structural analogs thereof, L₃ is chosen from —OC(O)—, —C(O)O—, —NHC(O)—, —C(O)NH—, —OC(O)NH—, —NHC(O)O—, and structural analogs thereof; L₄ and L₅ are independently optional and chosen from an alkyl group and an alkenyl group, and the like; R₂ and R₃ are independently optional, wherein the bioconjugation reagent comprises at least one R₂ and/or R₃, and chosen from —CO₂⁻, —CO₂H, —SO₃⁻, —SO₃H, —SO₂⁻, —PO₂H²⁻, —PO₃H⁻, and —PO₄H²⁻, polymeric groups comprising one or more of the anionic groups(s), and structural analogs thereof; n is independently at each occurrence 0, 1, 2, 3, 4, 5, or 6; and x is independently at each occurrence 1, 2, 3, 4, 5, or 6. In various examples, the bioconjugation reagent comprises (or consists of) the following structure:

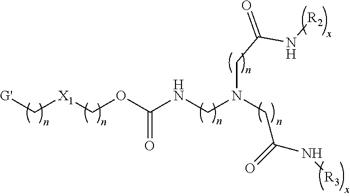

or a structural analog thereof or a pharmaceutically acceptable salt, a salt, a partial salt, a solvate, a polymorph, a prodrug, or the like thereof, or a stereoisomer or a mixture of stereoisomers, an isotopic variant, a tautomer, or the like thereof. In various examples, the bioconjugation reagent comprises (or consists of) the following structure:

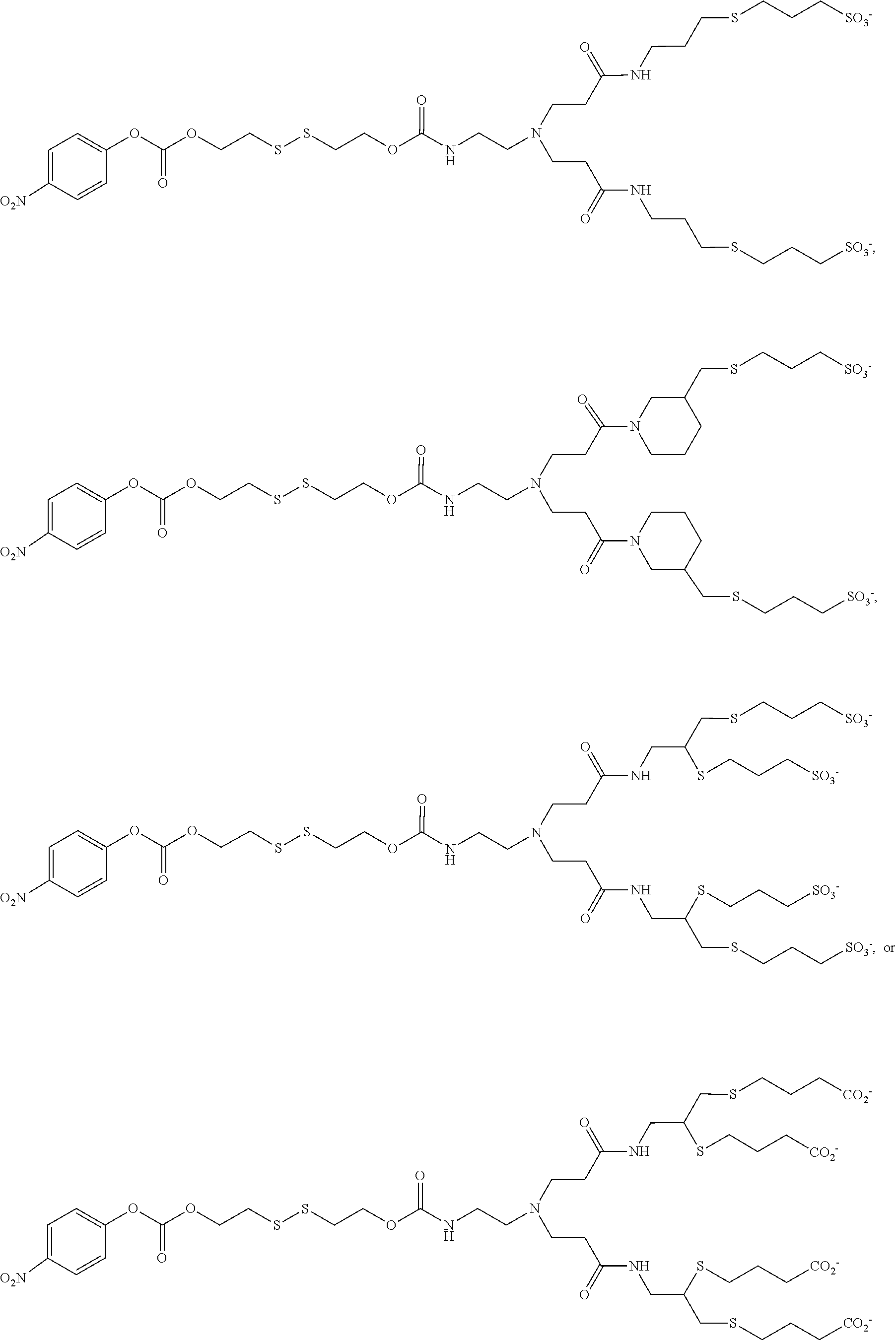

or a structural analog thereof or a pharmaceutically acceptable salt, a salt, a partial salt, a solvate, a polymorph, a prodrug, or the like thereof, or a stereoisomer or a mixture of stereoisomers, an isotopic variant, a tautomer, or the like thereof. In various examples, the conjugation group is chosen from carbonate groups and ester groups, and structural analogs thereof. In various examples, the conjugation group is chosen from N-hydroxysuccinimide ester groups, nitrophenol carbonate groups, pentafluorophenyl carbonate groups, trifluorophenyl carbonate groups, hexafluoropropanol carbonate groups, and trimethylaminophenyl carbonate groups, and structural analogs thereof. In various examples, the cleavable group comprises (or consists of) a stimuli-cleavable bond chosen from redox-cleavable disulfide bonds, light-cleavable bonds, ROS-cleavable bonds, and pH-cleavable bonds, and the like. In various examples, the stimuli-cleavable bond comprises (or consists of) a disulfide bond (e.g., a redox-cleavable disulfide bond or the like) or the like.

In various examples, a method of making one or more of the anionically functionalized polypeptide(s) (e.g., anionically functionalized polypeptide(s) of the present disclosure), comprising: forming a reaction mixture comprising one or more of the bioconjugation reagent(s) (e.g., bioconjugation reagents of the present disclosure), and one or more polypeptide(s); and, holding the reaction mixture (e.g., for a time and/or temperature), where the anionically functionalized polypeptide(s) is/are formed. In various examples, the one or more polypeptide(s) are chosen from enzymes, receptor ligands, transcriptional factors, growth factors, antibodies, peptide or protein immunogens, protein-based therapeutic agents, toxins, cytokines, hormones, fluorescent proteins, any fragments thereof, and structural analogs thereof, and the like, and any combination thereof. In various examples, the enzymes are chosen from Clustered Regularly Interspaced Short Palindromic Repeats (CRISPR) enzymes and structural analogs thereof, and the like. and structural analogs thereof, and the like. In various examples, at least a portion or all of the one or more polypeptide(s) is/are modified polypeptide(s) or the like. In various examples, the method further comprises modifying at least a portion or all of the anionically functionalized polypeptide(s). In various examples, at least a portion of the one or more polypeptide(s) each react with one or more conjugation group(s) of the one or more bioconjugation reagent(s), wherein the peptide group(s) and the conjugated group(s) are formed. In various examples, the reaction mixture further comprises one or more solvent(s).

### BRIEF DESCRIPTION OF THE FIGURES

For a fuller understanding of the nature and objects of the disclosure, reference should be made to the following detailed description taken in conjunction with the accompanying figures.

FIG. 1 shows a schematic of the bioreversible anionic cloaking strategy. Chemical modification of surface-exposed lysines with sulfonated cloaking reagents can enable complexation and subsequent delivery of protein cargos with cationic lipids. Following endocytic escape, the reagents can be cleaved off via the presence of a self-immolative, redox-sensitive disulfide bond to tracelessly deliver the cargo protein.

FIG. 2 shows conjugation of sfGFP with lysine-reactive sulfonated probes results in anionic modification. a, Panel of sulfonated p-nitrophenyl carbonate compounds synthesized. b, Native gel electrophoresis of sfGFP samples conjugated to sulfonated compounds, before and after treatment with 10 mM GSH. c, Isoelectric focusing gels of sfGFP conjugated to sulfonated compounds. d, MALDI spectra of sfGFP samples conjugated to sulfonated compounds (modified with 30 molar equivalents).

FIG. 3 shows delivery of anionically-cloaked sfGFP with Lipofectamine 2000 (LF2K). Transfections of sfGFP complexed with LF2K were performed at 500 nM into HEK293T cells for 6 hours. a, Representative flow cytometry histograms of HEK293T cells transfected with sfGFP and sfGFP modified with 30 molar equivalents of each sulfonated compound, using LF2K. b, Percent GFP-positive HEK293T cells following transfections of sfGFP and anionically-modified sfGFP (with molar equivalents of sulfonated compounds as indicated) using LF2K. c, Representative confocal microscopy images of HEK293T cells transfected with sfGFP and sfGFP-SL4 (modified with 30 molar equivalents of SL4) using LF2K. Scale bar=10 μm (μm=micron(s)). All data are presented as mean±SD (n=3 for flow cytometry). Statistical significance was determined by ordinary one-way ANOVA followed by Bonferroni correction for multiple comparisons (*p<0.05, **p<0.01, ***p<0.001, ****p<0.0001).

FIG. 4 shows delivery of anionically-cloaked sfGFP with MC3 LNPs. Transfections of sfGFP using MC3 LNPs were performed at 250 nM (nM=nanomolar) into HEK293T cells for 6 hours. Unless otherwise stated, data shown are for sfGFP cloaked with 30 molar equivalents of SL4 or CL4 and for MC3 LNPs (10 wt/wt, MC3/sfGFP) supplemented with 10 mol % DOTAP and formulated in pH 5 citrate buffer. a, Representative flow cytometry histograms of HEK293T cells transfected with sfGFP and sfGFP cloaked with SL4 or CL4, using MC3 LNPs. For comparison, HEK293T cells were transfected with 500 nM of the same sfGFP proteins complexed with LF2K. b, Percent GFP-positive HEK293T cells following transfections of sfGFP-SL4 and sfGFP-CL4 using LF2K and MC3 LNPs. c, Representative flow cytometry histograms of HEK293T cells transfected with 10-250 nM of sfGFP-SL4 using MC3 LNPs. d, Percent GFP-positive HEK293T cells following transfections of sfGFP-SL4 using MC3, ALC-0315, and SM-102 LNPs. LNPs (10 wt/wt, ionizable lipid/sfGFP) were supplemented with 10 mol % DOTAP and formulated in pH 5 citrate buffer. e, Viability of HEK293T cells following transfections of sfGFP alone and sfGFP-SL4 using LF2K and MC3 LNPs, as measured by MTS assay. f, Percent GFP-positive HEK293T cells following transfections of sfGFP-SL4 and sfGFP-CL4 using MC3 LNPs formulated in citrate buffers at pH 5 and pH 7.4. g, Representative confocal microscopy images of HEK293T cells transfected with sfGFP, sfGFP-SL4, and sfGFP-CL4 using MC3 LNPs. Scale bar=10 m. All data are presented as mean±SD (n=3 for flow cytometry; n=4 for MTS). Statistical significance was determined by unpaired t-tests followed by Bonferroni-Dunn correction for multiple comparisons (*p<0.05, **p<0.01, ***p<0.001, ****p<0.0001).

FIG. 5 shows delivery of anionically-cloaked RNase A with MC3 LNPs. Data shown are for MC3 LNPs (10 wt/wt, MC3/RNase A) supplemented with 10 mol % DOTAP and formulated in pH 5 citrate buffer. Unless otherwise stated, all RNase A transfections were performed for 48 hours. a, Schematic depicting RNase A delivery strategy. Following delivery, RNase A will induce degradation of intracellular RNA, leading to cell death. b, Ribonuclease activity of native RNase A and RNase A-SL4. Activity assays were repeated for RNase A samples incubated overnight with 10 mM DTT. c, Viability of HEK293T cells following 500 nM transfections of RNase A-SL4 using MC3 LNPs, as measured by MTS assay. RNase A was modified with 5-15 molar equivalents of SL4 either containing redox-cleavable disulfide bonds or non-cleavable butyl linker. d, Viability of cancer cell lines following transfections of RNase A-SL4 from 10 nM-1000 nM using MC3 LNPs, as measured by MITS assay. RNase A was modified with 10 molar equivalents of SL4. HeLa cells treated with blank MC3 LNPs (gray line) at equivalent lipid amounts served as a negative control. e, Half-maximal effective concentration of delivery, EC₅₀, vs IC₅₀ values for cells transfected with RNase A-SL4 using MC3 LnPs. RNase A was modified with 10 molar equivalents of SL4. Calculated IC₅₀ values are shown under each cell line. EC₅₀ values calculated by transfecting cells with 10-500 nM of fluorescein-labeled RNase A-SL4 using MC3 LNPs for 6 hours and quantifying percent positive fluorescein-RNase A-SL4 cells using flow cytometry. All data are presented as mean±SD (n=3 for flow cytometry; n=4 for MITS; n=4 for ribonuclease assay). Statistical significance was determined by two-way ANOVA followed by Bonferroni correction for multiple comparisons (*p<0.05, **p<0.01, ***p<0.001, ****p<0.0001).

FIG. 6 shows delivery of anionically-cloaked anti-β-catenin antibody with MC3 LNPs. Data shown are for MC3 LNPs (MC3/IgG, 2 wt/wt) supplemented with 10 mol % DOTAP and formulated at pH 5. a, Schematic of IgG delivery strategy. Binding of β-catenin by anti-β-catenin IgG will prevent association of β-catenin with TCF and inhibit expression of Wnt-driven genes. b, Binding activity of anti-β-catenin IgG to immobilized β-catenin as determined by ELISA in the presence or absence of DTT. IgGs were cloaked with 30 molar equivalents of SL4. c, Knockdown of TCF-driven TOPFlash luciferase activity following transfection of DLD1 cells with 50-500 nM anti-β-catenin IgG-SL4 and isotype IgG-SL4 using MC3 LNPs. DLD-1 cells transfected with FOPFlash plasmid, which contains mutated TCF sites upstream of luciferase expression cassette, served as a negative control. Transfections were performed for 24 hours. d, Percent of fluorescein-IgG positive DLD-1 cells following transfections of 50-500 nM fluorescein-labeled anti-β-catenin IgG-SL4 and isotype IgG-SL4 using MC3 LNPs for 6 hours. IgGs were cloaked with 30 molar equivalents of SL4. All data are presented as mean±SD (n=3 for flow cytometry; n=3 for ELISA; n=3 for TOPFlash assay). Statistical significance was determined by unpaired t tests and two-way ANOVA followed by Bonferroni correction for multiple comparisons (*p<0.05, **p<0.01, ***p<0.001, ****p<0.0001).

FIG. 7 shows in vivo biodistribution of anionically-cloaked mCherry with MC3 LNPs. Data shown are for mCherry cloaked with 30 molar equivalents of SL4. a, Heat map of LNP formulation optimization of anionically-cloaked mCherry. MC3 LNPs were formulated with mCherry-SL4 using varying amounts of PEG-DMG-2000 and DOTAP (total lipids/mCherry, 20 wt/wt) in pH 5 buffer. Size distribution, serum stability, transfection efficiency into HEK293T cells, and cellular cytotoxicity were measured for the nine formulations. b, Ex vivo fluorescent images of organs following tail vein injections into SKH1 mice of PBS, free mCherry, and mCherry-SL4 formulated in MC3 LNPs. MC3 LNPs were formulated with 3 mol % PEG-DMG-2000 and 30 mol % DOTAP. Injections were performed of 1 mg/kg of total protein for 1.5 hr (hr=hour(s)) and 24 hr. c, Quantified average radiant efficiency of ex vivo fluorescent images of harvested lungs, liver, and kidneys from SKH1 mice. All data are presented as mean±SD (n=3 for mice injections). Units are expressed in Radiant Efficiency (p/sec/cm²/sr)/(μW/cm²).

FIG. 8 shows representative flow cytometry histograms of sfGFP (modified with 30 molar eq. of SL4) transfections into HEK293T cells with Lipofectamine 2000 from 50 nM-500 nM.

FIG. 9 shows (left) in-gel fluorescence images of gels run under native conditions of sfGFP-SL4 (modified with 30 molar eq.) incubated in different pH buffers and in the presence of ethanol (1 ug protein/well). Indicated conditions are intended to reflect the conditions of LNP formulation. (right) Emission spectrum of sfGFP-SL4 at varying pH conditions.

FIG. 10 shows representative in-gel fluorescence image of gel electrophoresis run under native conditions of sfGFP-SL4 (modified with 30 molar eq.) formulated with MC3 LNPs (1 ug protein/well). LNPs were formulated in pH 5 and pH 7.4 buffers and supplemented with or without 10 mol % DOTAP. LNP samples were treated with Triton-X to dissolve LNPs and release encapsulated proteins.

FIG. 11 shows representative in-gel fluorescence image of gel electrophoresis run under native conditions of sfGFP-CL4 (modified with 30 molar eq.) formulated with MC3 LNPs (1 ug protein/well). LNPs were formulated in pH 5 and pH 7.4 buffers and supplemented with or without 10 mol % DOTAP. LNP samples were treated with Triton-X to dissolve LNPs and release encapsulated proteins.

FIG. 12 shows IEF gel of sfGFP conjugated to varying molar eq. of CL4 before and after incubation with 10 mM DTT.

FIG. 13 shows IEF gel of RNase A conjugated to varying molar eq. of SL4 before and after incubation with 10 mM DTT.

FIG. 14 shows MALDI spectra of RNase A and RNase A modified with varying molar eq. of SL4.

FIG. 15 shows CD spectra of native RNase A, RNase A-SL4 (modified with 10 molar eq. of SL4), and RNase-SL4 incubation with 10 mM DTT.

FIG. 16 shows RNase activity assay of native RNase A and RNase A modified with SL4 (n=4). RNase-SL4 samples were co-incubated with 10 mM of either GSH or DTT during the course of the assay.

FIG. 17 shows RNase activity assay of native RNase A and RNase A modified with non-cleavable SL4 (n=4). Activity assays were repeated for RNase A samples incubated overnight with 10 mM DTT.

FIG. 18 shows viability of HEK293T cells transfected with 500 nm of RNase A-SL4 as measured by MTS assay (n=4). RNase A was modified with SL4 containing redox-cleavable disulfide linker and formulated with MC3 LNPs (1-10 wt/wt, MC3/Rnase A) with 10 mol % DOTAP in pH 5 citrate buffer. RNase A transfections were performed for 48 hours.

FIG. 19 shows viability of HEK293T cells transfected with 500 nm of RNase A modified with non-cleavable SL4 as measured by MITS assay (n=4). RNase A was modified with non-cleavable SL4 containing butyl linker and formulated with MC3 LNPs (1-10 wt/wt, MC3/Rnase A) with 10 mol % DOTAP in pH 5 citrate buffer. RNase A transfections were performed for 48 hours.

FIG. 20 shows IEF gel of AlexaFluor488 secondary IgG conjugated to varying molar eq. of SL4 before and after incubation with 10 mM DTT.

FIG. 21 shows MALDI spectra of anti-β-catenin IgG and IgG modified with 30 molar eq. of SL4.

FIG. 22 shows the percent of IgG positive HEK293T cells following transfections of AlexaFluor488 secondary IgG conjugated to varying molar eq. of SL4 and formulated with MC3 LNPs, as analyzed by flow cytometry (n=3). Secondary IgG-SL4 was formulated with MC3 LNPs (2 wt/wt, MC3/IgG) with 10 mol % DOTAP in pH 5 citrate buffer. Transfections were performed for 6 hours.

### DETAILED DESCRIPTION OF THE DISCLOSURE

Although claimed subject matter will be described in terms of certain examples and embodiments, other examples and embodiments, including examples that do not provide all of the benefits and features set forth herein, are also within the scope of this disclosure. Various structural, logical, and process step changes may be made without departing from the scope of the disclosure.

As used herein, unless otherwise stated, “about”, “substantially”, or “the like”, when used in connection with a measurable variable (such as, for example, a parameter, an amount, a temporal duration, or the like) or a list of alternatives, is meant to encompass variations of and from the specified value including, but not limited to, those within experimental error (which can be determined by, e.g., a given data set, an art accepted standard, etc. and/or with, e.g., a given confidence interval (e.g. 90%, 95%, or more confidence interval from the mean), such as, for example, variations of +/−10% or less, +/−5% or less, +/−1% or less, and +/−0.1% or less of and from the specified value), insofar such variations in a variable and/or variations in the alternatives are appropriate to perform in the instant disclosure. As used herein, the term “about” may mean that the amount or value in question is the exact value or a value that provides equivalent results or effects as recited in the claims or taught herein. That is, it is understood that amounts, sizes, compositions, parameters, and other quantities and characteristics are not and need not be exact, but may be approximate and/or larger or smaller, as desired, reflecting tolerances, conversion factors, rounding off, measurement error, or the like, or other factors known to those of skill in the art such that equivalent results or effects are obtained. In general, an amount, size, composition, parameter, or other quantity or characteristic, or alternative is “about” or “the like,” whether or not expressly stated to be such. It is understood that where “about,” is used before a quantitative value, the parameter also includes the specific quantitative value itself, unless specifically stated otherwise.

Ranges of values are disclosed herein. The ranges set out a lower limit value and an upper limit value. Unless otherwise stated, the ranges include the lower limit value, the upper limit value, and all values between the lower limit value and the upper limit value, including, but not limited to, all values to the magnitude of the smallest value (either the lower limit value or the upper limit value) of a range. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “0.1% to 5%” should be interpreted to include not only the explicitly recited values of 0.1% to 5%, but also, unless otherwise stated, include individual values (e.g., 1%, 2%, 3%, and 4%) and the sub-ranges (e.g., 0.5% to 1.1%; 0.5% to 2.4%; 0.5% to 3.2%, and 0.5% to 4.4%, and other possible sub-ranges) within the indicated range. It is also understood (as presented above) that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about, it will be understood that the particular value forms a further disclosure. For example, if the value “about 10” is disclosed, then “10” is also disclosed.

Unless otherwise stated, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlett, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, unless otherwise stated, the term “alkyl group” refers to branched or unbranched hydrocarbon groups that include only single bonds between carbon atoms (not including substituent(s), if any). In various examples, an alkyl group is a C₁ to C₁₀ alkyl group (e.g., C₁, C₂, C₃, C₄, C₅, C₆, C₇, C₈, C₉, or C₁₀), including all integer numbers of carbons and ranges of numbers of carbons therebetween. In various examples, an alkyl group is a saturated group. In various examples, an alkyl group is a cyclic alkyl group, e.g., a monocyclic alkyl group or a polycyclic alkyl group or the like. Examples of alkyl groups include, but are not limited to, methyl groups, ethyl groups, propyl groups, butyl groups, isopropyl groups, tert-butyl groups, cyclohexyl groups, and adamantyl groups, and the like. In various examples, an alkyl group is unsubstituted or substituted with one or more substituent(s). Examples of substituents include, but are not limited to, various substituents such as, for example, halide groups (—F, —Cl, —Br, and —I), aliphatic groups (e.g., alkyl groups, alkenyl groups, alkynyl groups, and the like), halogenated aliphatic groups (e.g., trifluoromethyl group and the like), aryl groups, halogenated aryl groups, hydroxyl group, amine groups, nitro group, cyano groups, isocyano groups, silane groups (e.g., alkyl silane groups, aryl silane groups, alkyl/aryl silane groups, or the like), alkoxide groups, alcohol groups, ether groups, ketone groups, carboxylate groups, carboxylic acid groups, ester groups, amide groups, thioether groups, carbamate groups, carboxylic acid groups, and the like, and any combination thereof.

As used herein, unless otherwise indicated, the term “alkenyl group” refers to branched or unbranched hydrocarbon groups comprising one or more carbon-carbon (C—C) double bond(s). In various examples, an alkenyl group is a terminal alkenyl group (the C—C double bond is at an end of the hydrocarbon group) or an internal alkenyl group (the C—C double bond is not at an end of the hydrocarbon group). In various examples, an alkenyl group is a C₂ to C₁₀ alkenyl group, including all integer numbers of carbons and ranges of numbers of carbons therebetween (e.g., a C₂, C₃, C₄, C₅, C₆, C₇, C₈, C₉, or C₁₀ alkenyl group). In various examples, an alkenyl group is a cyclic alkenyl group, a polycyclic aliphatic group (e.g., an aliphatic group comprising a strained ring and/or bridging group), or the like (e.g., an exocyclic alkenyl group or an endocyclic alkenyl group). In various examples, an alkenyl group is conjugated or non-conjugated. In various examples, an alkenyl group is unsubstituted or substituted with one or more substituent(s). Examples of substituents include, but are not limited to, halide groups (—F, —Cl, —Br, and —I), aliphatic groups (e.g., alkyl groups, alkenyl groups, alkynyl groups, cycloaliphatic groups, and the like), halogenated aliphatic groups (e.g., trifluoromethyl group and the like), aryl groups, halogenated aryl groups, hydroxyl groups, (e.g., aliphatic alcohol groups, aliphatic diol groups, aliphatic polyol groups, and the like), amine groups, nitro groups, cyano groups, isocyano groups, silyl groups, alkoxide groups, ether groups, ketone groups, carboxylate groups, carboxylic acid groups, ester groups, amide groups, ether groups, thioether groups, and the like, and any combination thereof. In various examples, an aryl group substituent or substituents comprise(s) one or more heteroatom(s), such as, for example, oxygen, nitrogen, sulfur, and the like, and any combination thereof. Examples of alkenyl groups include, but are not limited to, an ethenyl (vinyl) group, 1-propenyl groups, 2-propenyl (allyl) groups, 1-, 2-, and 3-butenyl groups, isopropenyl groups, norbornenyl groups, cyclohexenyl groups, structural analogs thereof, and the like.

As used herein, “polypeptides” or “anionically functionalized polypeptides” refers to amino acid residue sequences. Those sequences are written left to right in the direction from the amino to the carboxy terminus. In various examples, a polypeptide comprises one or more canonical amino acid(s) (e.g., L- and/or D-enantiomers thereof), one or more non-canonical amino acid(s) (e.g., L- and/or D-enantiomers thereof), which independently may be an alpha-amino acid or a beta-amino acid, or any combination thereof. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V). Unless indicated otherwise, a “polypeptide” or an “anionically functionalized polypeptide” includes proteins and peptides. In various examples, polypeptide refers to a molecule comprising one or more chain(s) of amino acids in a specific order.

As used herein, unless otherwise stated, the term “structural analog” refers to any polypeptide or group that can be envisioned to arise from an original polypeptide, if one atom or group of atoms, functional groups, or substructures is replaced with another atom or group of atoms, functional groups, substructures, or the like. In various examples, the term “structural analog” refers to any group that is derived from an original any polypeptide by a chemical reaction, where the any polypeptide or group is modified or partially substituted such that at least one structural feature of the original any polypeptide is retained.

As used herein, unless otherwise stated, the term “group” refers to a chemical entity that is monovalent (i.e., has one terminus that can be (is) covalently bonded to other chemical species), divalent, or polyvalent (i.e., has two or more termini that can be (are) covalently bonded to other chemical species). The term “group” also includes radicals (e.g., monovalent radicals and multivalent radicals, such as, for example, divalent radicals, trivalent radicals, and the like). Illustrative examples of groups include:

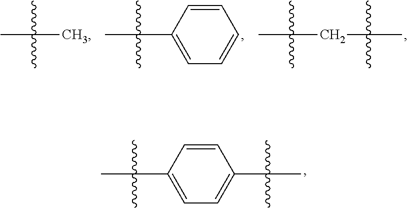

and the like.

The present disclosure provides, inter alia, polypeptides and methods of making anionically functionalized polypeptides. The present disclosure also provides uses of the anionically functionalized polypeptides.

In an aspect, the present disclosure provides polypeptides. In various examples, a polypeptide (e.g., a functionalized polypeptide, such as, for example, a functionalized protein, a functionalized peptide, or the like) comprises one or more anionic functional group(s), each anionic functional group comprising a conjugated group, a cleavable group, optionally, one or more linking group(s), and one or more anionic group(s). Such functional groups may be referred to as exogenous anionic functional groups. A polypeptide may be referred to in the alternative as a functionalized polypeptide, anionic-group functionalized polypeptide, anionically functionalized polypeptide, or an anionically modified polypeptide. In various examples, an anionically functionalized polypeptide is capable of intracellular transport (e.g., transport across the lipid bilayers of a cell or the like). In various examples, an anionically functionalized polypeptide is made by a method of the present disclosure. Non-limiting examples of polypeptides are described herein.

Peptide group(s) of an anionically functionalized polypeptide is/are not particularly limited. A peptide group can be a structural analog of (or be formed from) various polypeptides. In various examples, a peptide group comprises one or more lysine residue(s). In various examples, an anionically functionalized polypeptide comprises a peptide group formed from (or is a structural analog of) a naturally-occurring polypeptide, a non-naturally occurring polypeptide (such as, for example, a synthetic polypeptide, or the like), or the like. In various examples, an anionically functionalized polypeptide comprises a peptide group formed from (or is a structural analog of) a modified polypeptide. In various examples, an anionically functionalized polypeptide comprises a peptide group formed from (or is a structural analog of) a recombinant polypeptide. In various examples, an anionically functionalized polypeptide comprises a peptide group formed from (or is a structural analog of) an antibody, cytokine, hormone, fluorescent protein groups, or any fragment thereof, or the like. In various examples, an anionically functionalized polypeptide comprises a peptide group formed from (or is a structural analog of) a therapeutic polypeptide.

A peptide group can have various molecular weights. In various examples, a peptide group has a molecular weight of 1 kilodalton (kDa) to 300 kDa, including all 0.1 kDa values and ranges therebetween (e.g., 10 kDa to 200 kDa or 15 kDa to 115 kDa).

In various examples, a polypeptide forms (e.g., as a result of reaction of the cleavage group(s) of anionic functional group(s)) the native polypeptide (or substantially the native polypeptide), from which the peptide group was formed. In various examples, the reaction is an intracellular reaction.

An anionic functional group can comprise various anionic groups. An anionic group may be a protonated anionic group, a deprotonated anionic group, an anionic group salt, or the like. In various examples, an anionic group is capable of interacting with a cationic lipid (such as, for example, a cationic lipid of a cationic lipid reagent or the like). Non-limiting examples of anionic groups include sulfonate groups, carboxyl groups, carboxylate groups, sulfonic acid groups, sulfur dioxide groups, phosphinate groups, phosphonate, groups, phosphate groups, and the like, and any combination thereof. In various examples, the anionic group is a CO₂⁻ group, a CO₂H group, a SO₃⁻ group, a SO₃H group, a SO₂⁻ group, a PO₂H²⁻ group, a PO₃H⁻ group, a PO₄H²⁻ group, or a polymeric group comprising one or more of the anionic groups(s), or the like.

An anionically functionalized polypeptide comprising one or more anionic functional group(s) can comprise various numbers of anionic functional group(s). In various examples, at least a portion of the sidechains (such as, for example, a nucleophilic amino acid sidechain group or groups, a lysine sidechain group or groups, an ornithine sidechain group or groups, a serine sidechain group or groups, a threonine sidechain group or groups, a histidine sidechain group or groups, a methionine sidechain group or groups, or the like) or the N-terminus group, or any combination thereof of the polypeptide is functionalized with an anionic functional group. In various examples, at least a portion of the sidechains or N-terminus groups of the polypeptide are functionalized with an anionic functional group, such that the anionically functionalized polypeptide is capable of intracellular delivery (e.g., transport of the anionically functionalized polypeptide across the lipid bilayers of a cell or the like). In various examples, 1 to 25 mol percent, including all 0.1 mol percent values and ranges therebetween, (e.g., 1, 2, 3, 4, 5, 6, 7, 8, 9, 10, 11, 12, 13, 14, 15, 16, 17, 18, 19, 20, 21, 22, 23, 24, or 25) of the sidechains (such as, for example, a nucleophilic amino acid sidechain group or groups, a lysine sidechain group or groups, an ornithine sidechain group or groups, a serine sidechain group or groups, a threonine sidechain group or groups, a histidine sidechain group or groups, a methionine sidechain group or groups, or the like, or any combination thereof, if present) and/or N-terminus groups of the anionically functionalized polypeptide are functionalized with an anionic functional group.

In various examples an anionically functionalized polypeptide further comprises one or more other exogenous group(s) In various examples, the exogenous group(s) facilitate(s) transport of the polypeptide across the lipid bilayer of a cell. Examples of suitable groups and reagents/methods for forming such groups are known in the art. Non-limiting examples of other exogenous groups include lipid-based reagents and/or polymer-based lipid reagents, other groups that facilitate transport of the polypeptide across the lipid bilayer of a cell, targeting groups, tags/reporters, and the like, and any combination thereof.

As used herein, “targeting group” refers to molecules, complexes, agents, and the like that are capable of specifically or selectively interacting with, binding with, acting on or with, or otherwise associating or recognizing a target molecule, agent, and/or complex that is associated with, part of, coupled to, another object, complex, surface, and the like, such as, for example, a cell or cell population, tissue, organ, subcellular locale, object surface, particle, or the like. Nonlimiting examples of targeting groups include chemical, biological, metals, polymers, other agents and molecules with targeting capabilities, and the like. Other non-limiting examples of targeting groups include amino acids, peptides, polypeptides, nucleic acids, polynucleotides, lipids, sugars, metals, small molecule chemicals, and the like, and any combination thereof. Other non-limiting examples of targeting groups are antibodies or fragments thereof, aptamers, affibodies, avimers, DNA, RNA (such as, for example, guide RNA for a RNA guided nuclease or system), ligands, substrates, enzymes, and the like, and any combination thereof. The specificity or selectivity of a targeting group can be determined by any suitable method or technique that will be appreciated by those of ordinary skill in the art. For example, in various examples, the methods described herein include determining the disassociation constant for the targeting group and target. In various examples, the targeting group has a specificity the equilibrium dissociation constant, K_{d}, is 10⁻³ M or less, 10⁻⁴ M or less, 10⁻⁵ M or less, 10⁻⁶ M or less, 10⁻⁷ M or less, 10⁻⁸ M or less, 10⁻⁹ M or less, 10⁻¹⁰ M or less, 10⁻¹¹ M or less, or 10⁻¹² M or less under the conditions employed, e.g., under physiological conditions such as, for example, those inside a cell or consistent with cell survival. In various examples, specific binding can be accomplished by a plurality of weaker interactions (e.g., a plurality of individual interactions, wherein each individual interaction is characterized by a K_{d} of greater than 10⁻³ M). In various examples, the targeting group has increased binding with, association with, interaction with, activity on as compared to non-targets, such as a 1 to 500 or more fold increase. In various examples, targets of targeting groups are amino acids, peptides, polypeptides, nucleic acids, polynucleotides, lipids, sugars, metals, small molecule chemicals, or the like, or any combination thereof. In various examples, targets comprise receptors, biomarkers, transporters, antigens, complexes, and the like, and any combination thereof. In various examples, targets are internal and/or external (i.e., expressed on the surface of a cell) to cells. Exemplary target cells include, but are not limited to liver cells, pancreatic cells, muscle cells (e.g., skeletal, cardiac, and/or smooth muscle cells), brain cells, neurons, nerve support cells (e.g., glial cells, Schwann cells, astrocytes, dendrites, etc.), immune cells (T-cells, B-cells, monocytes, macrophages, dendritic cells, NK cells, neutrophils, plasma cells, etc.), kidney cells, thyroid cells, bone cells, gastrointestinal tract cells, auditory cells (e.g., hair cells), eye cells (e.g., retinal cells, corneal cells, etc.), skin cells, lung cells, adipocytes, bladder cells, olfactory cells, vasculature cells, cancer cells, tumor cells, cancer stem cells, or the like, or any combination thereof. In various examples, the target cells are diseased or the like. In various examples, the target cells are normal (non-diseased) or the like. In various examples, the target cells are progenitor cells or the like. In various examples, the target cells are differentiated cells or the like.

In various examples, an anionically functionalized polypeptide further comprises a reporter molecule and/or tag operatively coupled the polypeptide. Nonlimiting example of reporter molecules and tags include affinity tags, such as, for example, chitin binding protein (CBP), maltose binding protein (MBP), glutathione-S-transferase (GST), poly(His) tag; solubilization tags such as, for example, thioredoxin (TRX) and poly(NANP), MBP, and GST; chromatography tags such as, for example, those comprising a polyanionic amino acids, such as FLAG-tag; epitope tags such as, for example, V5-tag, Myc-tag, HA-tag and NE-tag; protein tags that can allow specific enzymatic modification (such as biotinylation by biotin ligase) or chemical modification (such as reaction with FlAsH-EDT2 for fluorescence imaging), DNA and/or RNA segments that contain restriction enzyme or other enzyme cleavage sites; DNA segments that encode products that provide resistance against otherwise toxic compounds including antibiotics, such as spectinomycin, ampicillin, kanamycin, tetracycline, Basta, neomycin phosphotransferase II (NEO), hygromycin phosphotransferase (HPT) and the like; DNA and/or RNA segments that encode products that are otherwise lacking in the recipient cell (e.g., tRNA genes, auxotrophic markers); DNA and/or RNA segments that encode products which can be readily identified (e.g., phenotypic markers such as β-galactosidase, GUS); fluorescent proteins such as green fluorescent protein (GFP), cyan fluorescent protein (CFP), yellow fluorescent protein (YFP), red fluorescent protein (RFP), mCherry, or other optically active proteins e.g., luciferase, and cell surface proteins); optically active dyes (e.g., fluorescent, UV, IR, and NIR dyes), polynucleotides that can generate one or more new primer sites for PCR (e.g., the juxtaposition of two DNA sequences not previously juxtaposed), DNA sequences not acted upon or acted upon by a restriction endonuclease or other DNA modifying enzyme, chemical, etc.; epitope tags (e.g. GFP, FLAG- and His-tags), and, DNA sequences that make a molecular barcode or unique molecular identifier (UMI), DNA sequences required for a specific modification (e.g., methylation) that allows its identification. Other suitable markers will be appreciated by those of skill in the art.

In various examples, an anionically functionalized polypeptide comprises one or more nuclear localization signals (NLSs) at the C-terminus, the N-terminus, or both the N- and C-terminus of the polypeptide. Without being bound by theory, such sequences may increase the transport of the polypeptide to the nucleus of a cell. In various examples, the NLSs used in the context of the present disclosure are heterologous to the polypeptide. Non-limiting examples of NLSs include an NLS sequence derived from: the NLS of the SV40 virus large T-antigen, having the amino acid sequence PKKKRKV (SEQ ID NO: 1) or PKKKRKVEAS (SEQ ID NO: 2); the NLS from nucleoplasmin (e.g., the nucleoplasmin bipartite NLS with the sequence KRPAATKKAGQAKKKK (SEQ ID NO: 3)); the c-myc NLS having the amino acid sequence PAAKRVKLD (SEQ ID NO: 4) or RQRRNELKRSP (SEQ ID NO: 5); the hRNPA1 M9 NLS having the sequence NQSSNFGPMKGGNFGGRSSGPYGGGGQYFAKPRNQGGY (SEQ ID NO: 6); the sequence RMRIZFKNKGKDTAELRRRRVEVSVELRKAKKDEQILKRRNV (SEQ ID NO: 7) of the IBB domain from importin-alpha; the sequences VSRKRPRP (SEQ ID NO: 8) and PPKKARED (SEQ ID NO: 9) of the myoma T protein; the sequence PQPKKKPL (SEQ ID NO: 10) of human p53; the sequence SALIKKKKKMAP (SEQ ID NO: 11) of mouse c-abl IV; the sequences DRLRR (SEQ ID NO: 12) and PKQKKRK (SEQ ID NO: 13) of the influenza virus NS1; the sequence RKLKKKIKKL (SEQ ID NO: 14) of the Hepatitis virus delta antigen; the sequence REKKKFLKRR (SEQ ID NO: 15) of the mouse Mx1 protein; the sequence KRKGDEVDGVDEVAKKKSKK (SEQ ID NO: 16) of the human poly(ADP-ribose) polymerase; and the sequence RKCLQAGMNLEARKTKK (SEQ ID NO: 17) of the steroid hormone receptors (human) glucocorticoid, TAT, and R10, or any combination thereof. Additional NLSs that are suitable for use with the present disclosure as described herein are any of those in Srivaths et al. Bioinformation 2018, 14(3), 132; Physiol. Res. 67 (Suppl. 2): S267-S279, 2018; and Lange et al. J. Biol. Chem. 2007, 282(8), 5101-5105.

In various examples, an anionically functionalized polypeptide comprises groups that or are further modified to modify and/or optimize one or more characteristic(s), function(s), or activitie(s) of the polypeptide. In various examples, the modification modifies protein stability, modify half-life, improve storageability, optimize immunogenicity, optimize trafficking, optimize protein-protein interactions, modify activity, or the like, or any combination thereof. In various examples, the modified proteins are reversibly modified. In various examples, the modification is a post-translational modification (PTM), post-synthesis modification, or the like. In various examples, the modification is an amino acid side chain modification of peptide after its synthesis. There are more than 400 different types of PTMs affecting many aspects of protein functions, which can be applied to the modified polypeptides of the present disclosure. See e.g., Ramazi and Zahiri. Database (Oxford). 2021; 2021: baab012.

In various examples, an anionically functionalized polypeptide comprises groups that or are further modified to reduce aggregation. In various examples, the modified polypeptides are coupled to or otherwise associated with one or more cyclodextrins. See e.g., Serno et al., Adv Drug Deliv Rev. 2011 October; 63(13):1086-106, which can be adapted for use with the present modified polypeptides.

In various examples, an anionically functionalized polypeptide comprises groups that or are further modified modified with one or more lipids. See e.g., Nadolski and Linder. FEBS J. 2007 October; 274(20):5202-10, which can be adapted for use with the present modified polypeptides. In various examples, the modified proteins are modified by S-palmitoylation, which is a reversible posttranslational lipid modification of proteins. In various examples, the modified polypeptides are glycosylated at one or more residue(s). In various examples, the glycosylation includes N-linked glycosylation, O-linked glycosylation, or both. See e.g., Sola and Griebenow. BioDrugs. 2010; 24(1): 9-21; Delobel. Glycosylation of Therapeutic Proteins: A Critical Quality Attribute in Mass Spectrometry of Glycoproteins pp 1-21 partr of the Methods in Molecular Biology Series, volume 2271; Gupta and Shukla. Applied Microbiology and Biotechnology volume 102, pages 10457-10468 (2018); an Ma et al., Front. Chem., 23 Jul. 2020. Sec. Chemical Biology, which can be adapted for use with the present modified polypeptides.

In various examples, an anionically functionalized polypeptide comprises groups that or are further modified the modified polypeptide is phosphorylated at one or more residue(s). See e.g., Oza et al., Nature Communications volume 6, Article number: 8168 (2015), which can be adapted for use with the present modified polypeptides.

In various examples, an anionically functionalized polypeptide is acetylated at one or more residue(s). In various examples, the acetylation is Na-acetylation, Ne-acetylation, and/or O-acetylation. See e.g., Allfrey, V. G., Faulkner, R. and Mirsky, A. (1964) Acetylation and methylation of histones and their possible role in the regulation of RNA synthesis. Proc. Natl. Acad. Sci. U.S.A., 51, 786; Xia, C., Tao, Y., Li, M. et al. (2020) Protein acetylation and deacetylation: an important regulatory modification in gene transcription. Exp. Ther. Med., 20, 2923-2940; Huang, K.-Y., Lee, T.-Y., Kao, H.-J. et al. (2018) dbP™ in 2019: exploring disease association and cross-talk of post-translational modifications. Nucleic Acids Res., 47, D298-D308, which can be adapted for use with the present modified polypeptides.

In various examples, an anionically functionalized polypeptide is ubiquitinated at one or more residue(s). See e.g., Goldstein, G., Scheid, M., Hammerling, U. et al. (1975) Isolation of a polypeptide that has lymphocyte-differentiating properties and is probably represented universally in living cells. Proc. Natl. Acad. Sci., 72, 11-15; Lecker, S. H., Goldberg, A. L. and Mitch, W. E. (2006) Protein degradation by the ubiquitin-proteasome pathway in normal and disease states. J. Am. Soc. Nephrol., 17, 1807-1819; Swatek, K. N. and Komander, D. (2016) Ubiquitin modifications. Cell Res., 26, 399-422; Swatek, K. N. and Komander, D. (2016) Ubiquitin modifications. Cell Res., 26, 399-422, which can be adapted for use with the present modified polypeptides.

In various examples, an anionically functionalized polypeptide is SUMOylated at one or more residue(s). See e.g., Ramazi, S., Zahiri, J., Arab, S. et al. (2016) Computational prediction of proteins sumoylation: a review on the methods and databases. J. Nanomed. Res., 3, 00068, which can be adapted for use with the present modified polypeptides.

In various examples, an anionically functionalized polypeptide is myristoylated at one or more residue(s). See e.g., Wright, M. H., Heal, W. P., Mann, D. J. et al. (2010) Protein myristoylation in health and disease. J. Chem. Biol., 3, 19-35; Sedek, M. and Strous, G. J. (2013) SUMOylation is a regulator of the translocation of Jak2 between nucleus and cytosol. Biochem. J., 453, 231-239; Jentsch, S. and Psakhye, I. (2013) Control of nuclear activities by substrate-selective and protein-group SUMOylation. Annu. Rev. Genet., 47, 167-186; Mahajan, R., Delphin, C., Guan, T. et al. (1997) A small ubiquitin-related polypeptide involved in targeting RanGAP1 to nuclear pore complex protein RanBP2. Cell, 88, 97-107, which can be adapted for use with the present modified polypeptides.

In various examples, an anionically functionalized polypeptide is methylated at one or more residue(s). See e.g., Li, K. K., Luo, C., Wang, D. et al. (2012); Chemical and biochemical approaches in the study of histone methylation and demethylation. Med. Res. Rev., 32, 815-867, which can be adapted for use with the present polypeptides. In various examples, an anionically functionalized polypeptide is prenylated at one or more residues. In various examples, an anionically functionalized polypeptide is sulfonated at one or more residue(s). In various examples, the sulfation is N-sulfation and/or O-sulfation.

In various examples, an anionically functionalized polypeptide is chemically modified. See e.g., Sakamoto and Hamachi. Anal Sci. 2019 Jan. 10; 35(1):5-27; Boutureira and Bernades. Chem. Rev. 2015, 115, 5, 2174-2195; Spicer and Davis. Nature Communications volume 5, Article number: 4740 (2014); and Naowarojna et al. (2021) Synthetic and Systems Biol.) 6:32-49, which can be adapted for use with the present modified polypeptides. Protein chemical modification approaches can be roughly classified into three categories: 1) modifications via the reactivities of canonical (cAAs); 2) ribosomal-mediated incorporation of noncanonical amino acids (ncAAs); 3) modifications via affinity-driven ligand-directed reactions. In various examples, the modified polypeptide is modified at one or more Lys, Cys, Tyr, Trp, or any combination thereof. Lys is a convenient nucleophilic handle for many reactions. Site-selective Lys modification can be achieved by harnessing the pKₐ differences among various Lys residues. The thiol of Cys can be modified using many different reactions, e.g., alkylation and thiol-ene chemistry. The Tyr sidechain may exist in a phenol or a phenolate form. This allows selective modification by controlling the pH of the reaction. Common reactions include diazonium couplings and alkylation via π-allylpalladium complexes. The Trp indole group offers an opportunity for selective modification via metal-mediated C—H functionalization reaction. Exemplary modifications and reactions are set forth in e.g., Naowarojna et al., Synth Syst Biotechnol. 2021 March; 6(1): 32-49, which can be adapted for use with the present modified polypeptides.

In various examples, an anionically functionalized polypeptide is modified (such as, for example, engineered or the like) to contain one or more ncAAs. Incorporating ncAAs into proteins has been widely applied in biocatalysis to enhance the activity and selectivity of enzymes, and even allows researchers to obtain novel catalytic reactions that are naturally unavailable. See e.g., Drienovski I., Alonso-Cotchico L., Vidossich P., Lledós A., Marechal J.-D., Roelfes G. Design of an enantioselective artificial metallo-hydratase enzyme containing an unnatural metal-binding amino acid. Chem Sci. 2017; 8(10):7228-7235; rienovski I., Roelfes G. Expanding the enzyme universe with genetically encoded unnatural amino acids. Nat Catal. 2020:1-10; and Won Y., Pagar A. D., Patil M. D., Dawson P. E., Yun H. Recent advances in enzyme engineering through incorporation of unnatural amino acids. Biotechnol Bioproc Eng. 2019; 24(4):1-13, which can be adapted for use with the present modified polypeptides. In various examples, ncAAs are incorporated via genetic code expansion of the encoding polynucleotide. See e.g., Munier R., Cohen G. Incorporation of structural analogues of amino acids into bacterial proteins during their synthesis in vivo. Biochim Biophys Acta. 1959; 31(2):378; Dumas A., Lercher L., Spicer C. D., Davis B. G. Designing logical codon reassignment-Expanding the chemistry in biology. Chem Sci. 2015; 6(1):50-69; Bryson D. I., Fan C., Guo L.-T., Miller C., Söll D., Liu D. R. Continuous directed evolution of aminoacyl-tRNA synthetases. Nat Chem Biol. 2017; 13(12):1253; Drienovská I., Roelfes G. Expanding the enzyme universe with genetically encoded unnatural amino acids. Nat Catal. 2020:1-10, which can be adapted for use with the present modified polynucleotides and polypeptides.

Functional groups can be added to the polypeptide by bioorthagonal reactions at one or more residues (whether cAA or ncAA). Exemplary functional groups include, without limitation, alkynes, alkynes, alkenes, azides. and tetrazines, and/or the like. See e.g., Sakamoto S., Hamachi I. Recent progress in chemical modification of proteins. Anal Sci. 2018; 35(1) and Lang K., Chin J. W. Cellular incorporation of unnatural amino acids and bioorthogonal labeling of proteins. Chem Rev. 2014; 114(9):4764-4806, which can be adapted for use with the present modified polynucleotides and polypeptides.

In various examples, an anionically functionalized polypeptide is a modified anionically functionalized polypeptide. In various examples, a modified anionically functionalized polypeptide is formed from a modified polypeptide. In various examples, a modified anionically functionalized polypeptide is formed by modification of an anionically functionalized polypeptide. In various examples, an anionically functionalized polypeptide comprises a modification described herein. In various examples, an anionically functionalized polypeptide comprises one or more cyclodextrin group(s), comprise one or more lipid(s), is acetylated at one or more anionically functionalized polypeptide residue(s), is ubiquitinated at one or more anionically functionalized polypeptide residue(s), is myristoylated at one or more anionically functionalized polypeptide residue(s), is methylated at one or more anionically functionalized polypeptide residue(s), is chemically modified at one or more anionically functionalized polypeptide residue(s), or the like, or any combination thereof.

In various examples, an anionically functionalized polypeptide comprises the following structure: D-G-X-L-(R)ₓ. D comprises (or is) a peptide group; G comprises (or is) a conjugated group; X comprises (or is) a cleavable group; L comprises (or is) a linking group; and R comprises (or is) an anionic group. In various examples, x is 1, 2, 3, 4, 5, or 6.

Peptide group(s) of an anionically functionalized polypeptide is/are not particularly limited. In various examples, a peptide group is a functional peptide group (or formed from a functional polypeptide) or the like, or a fragment thereof. Non-limiting examples of peptide groups include enzyme groups (groups formed from an enzyme), receptor ligand (groups formed from a receptor ligand), transcriptional factor groups (or groups formed from a transcriptional factor), growth factor groups (or groups formed from a growth factor), antibody groups (or groups formed from an antibody or antigen-binding fragment thereof, such as, for example, a single-chain antibody fragment or Fab or the like), peptide or protein immunogens (e.g., that can be used for stimulating an immune response (e.g., a vaccine or the like) or the like), protein-based therapeutic agent groups (such as for example, protein-based chemotherapeutic agent groups or the like) (or groups formed from a protein-based therapeutic agent, such as for example, a protein-based chemotherapeutic agent or the like), toxin groups (or groups formed from a toxin), cytokine groups (or group formed from a cytokine), hormone groups (or groups formed from a hormone), fluorescent protein groups (or groups formed from a fluorescent protein), and the like, any fragments thereof, and any combination thereof. In various examples, an anionically functionalized polypeptide does not comprise a protein genetically fused with the polypeptide.

In various examples, a peptide group comprises (or is) a Clustered Regularly Interspaced Short Palindromic Repeats (CRISPR) enzyme group (or a group formed from any type of Clustered Regularly Interspaced Short Palindromic Repeats (CRISPR) enzyme or the like), including, but not limited to, Cas protein groups (or groups formed from any Cas protein (such as, for example, single effector Cas proteins, including, but not limited to, those with enzymatic activity and those that are enzymatically dead, including, but not limited to, Cas9, Cas12a, and Cas13, and the like) and the like. In various examples, a peptide comprises (or is) a reverse transcriptase group (or a group formed from a reverse transcriptase or any portion thereof, which may be used, for example, in prime editing or the like. In various examples, a peptide group comprises a cellular localization domain (e.g., so that the polypeptide formed from the anionically functionalized polypeptide can be trafficked to a cellular location, such as, for example, an organelle or the like), a nuclear localization signal, or the like, or both. In various examples, the anionically functionalized polypeptide is used for gene editing or otherwise modulating gene expression, or the like.

An anionically functionalized polypeptide can comprise various conjugated groups. In various examples, the conjugated group comprises (or consists of) the following structure

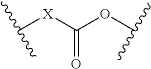

or a structural analog thereof, where X is chosen from an N group, an S group, an O group, and the like. In various examples, X is derived from the sidechain of an amino acid residue of the peptide.

An anionically functionalized polypeptide can comprise various cleavable groups. In various examples, a cleavable group comprises (or is) one or more stimuli-cleavable bond(s) (e.g., redox-cleavable disulfide bond(s) or the like), one or more light-cleavable bond(s), one or more ROS-cleavable bond(s), one or more pH-cleavable bond(s), or the like, or any combination thereof. In various examples, a cleavable group comprises (or is) one or more disulfide bond(s). In various examples, a cleavable group is chosen from stimuli-cleavable bonds (e.g., redox-cleavable disulfide bonds or the like), light-cleavable bonds, ROS-cleavable bonds, pH-cleavable bonds, and the like, and any combination thereof. In various examples, a cleavable group further comprises one or more linking group(s). In various examples, a linking group is an alkyl group or the like. In various examples, the cleavable group comprises (or consists of) the following structure

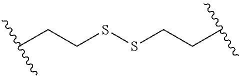 [SMILES: *CCSSCC*]

or a structural analog thereof.

An anionically functionalized polypeptide can comprise various linking groups. In various examples, an anionically functionalized polypeptide comprises one or more linking group(s). In various examples, the linking group(s) is/are independently chosen from —OC(O)—, —C(O)O—, —NHC(O)—, —C(O)NH—, —OC(O)NH—, and —NHC(O)O—, and the like. In various examples, a linking group comprises a branching point, such as, for example,

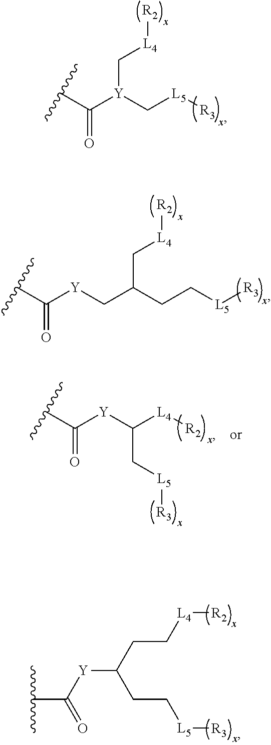

or a structural analog thereof.

In various examples, an anionically functionalized polypeptide comprises a peptide group covalently bonded to a conjugated group, the conjugated group covalently bonded to a cleavable group, and the cleavable group covalently bonded to one or more anionic group(s). The covalent bonds may include linking groups between, for example, the conjugated group and the cleavable group, or between the cleavable group and the anionic group(s). In various examples, the cleavable group is β- to the conjugated group. As an illustrative example, a disulfide bond of a cleavable group is β- to a carbamate group of a conjugated group.

In various examples, an anionically functionalized polypeptide comprises (or consists of) the following structure:

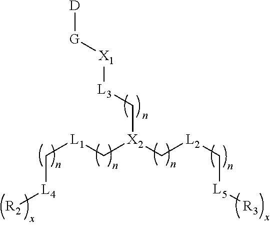

or a structural analog thereof. D comprises (or is) a peptide group; G comprises (or is) a conjugated group; X₂ comprises (or is) a cleavable group; X₁ is chosen from an N group, a CH group, a P group, and a P═O group, and the like; L₁ and L₂ are independently chosen from

### —OC(O)—, —C(O)O—, —NHC(O)—, —C(O)NH—, —OC(O)NH—, —NHC(O)O—,

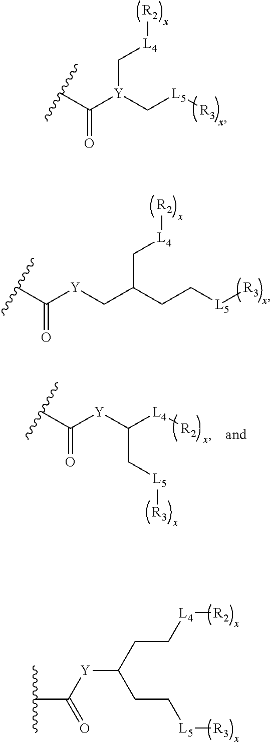

and structural analogs thereof, where Y is independently chosen from an NH group, an O group, an S group, a CH₂ group, an H group, a CH group, and the like; L₃ is chosen from
—OC(O)—, —C(O)O—, —NHC(O)—, —C(O)NH—, —OC(O)NH—, —NHC(O)O—, and structural analogs thereof, L₄ and L₅ are independently optional and chosen from alkyl groups, alkenyl groups, and the like, and R₂ and R₃ are independently optional, wherein the polypeptide comprises at least one R₂ and/or R₃, and are chosen from —CO₂⁻, —CO₂H, SO₃⁻, —SO₃H, —SO₂⁻, —PO₂H²⁻—, —PO₃H⁻, —PO₄H²⁻, and the like; and the like. In various examples, n is independently at each occurrence 0, 1, 2, 3, 4, 5, or 6. In various examples, x is independently at each occurrence 1, 2, 3, 4, 5, or 6.

In various examples, an anionically functionalized polypeptide comprises (or consists of) the following structure:

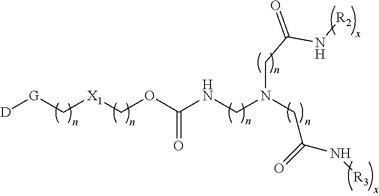

or structural analogs thereof.

In various examples, an anionically functionalized polypeptide comprises (or consists of) the following structure:

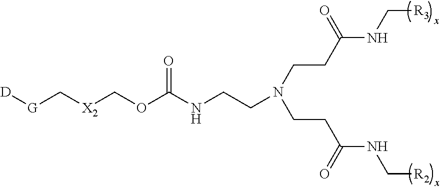

or structural analogs thereof.

In various examples, an anionically functionalized polypeptide comprises the following structure:

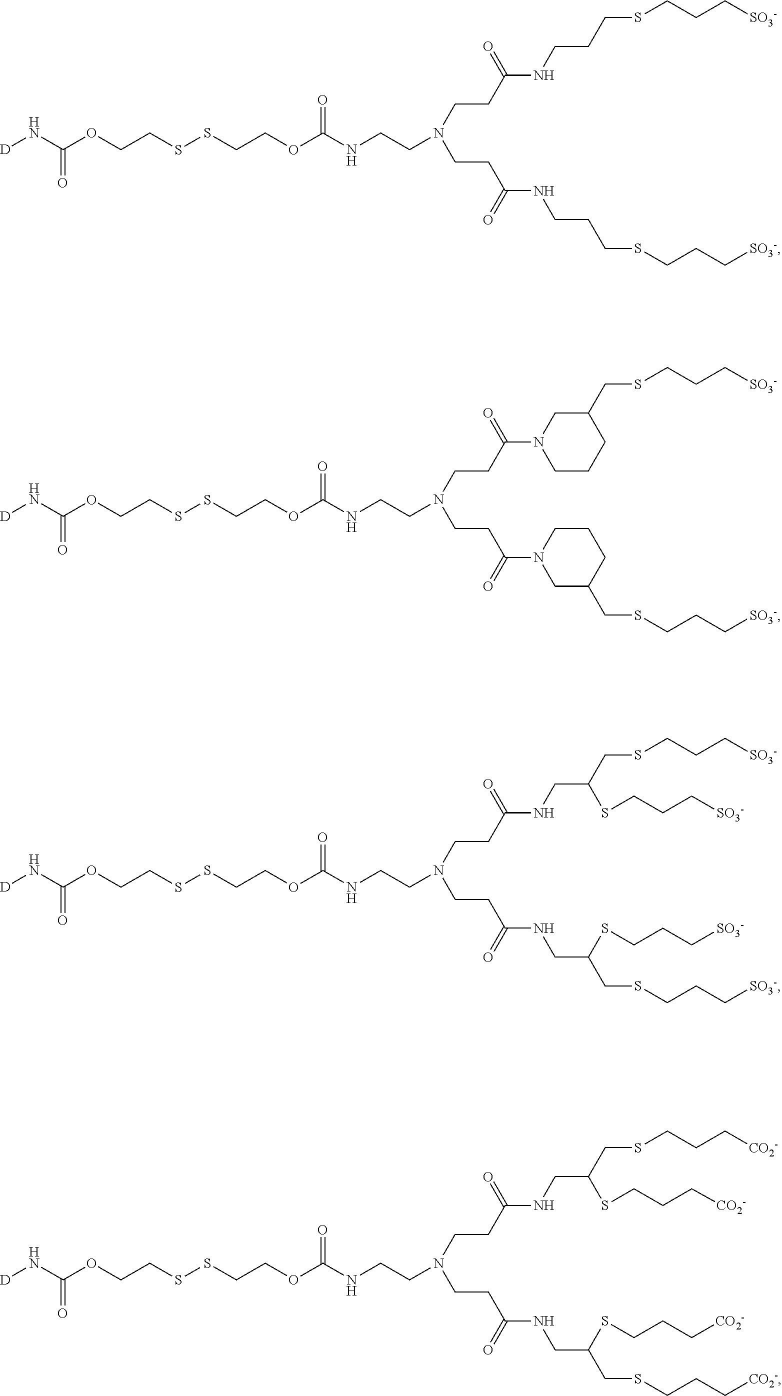

or a structural analog thereof.

In an aspect, the present disclosure provides compositions comprising one or more anionically functionalized polypeptide(s) (e.g., protein(s) and/or peptide(s)) of the present disclosure. A composition may be a pharmaceutical composition. Non-limiting examples of compositions are described herein.

A composition may comprise (or consist essentially of or consist of) one or more one or more anionically functionalized polypeptide(s) (e.g., protein(s) and/or peptide(s)). A composition may also comprise one or more additional component(s), one or more or all of which may be pharmaceutically acceptable components. In various examples, a composition comprises an anionically functionalized polypeptide, a structural analog thereof or a pharmaceutically acceptable salt, a salt, a partial salt, a solvate, a polymorph, a prodrug, or the like thereof, or a stereoisomer or a mixture of stereoisomers, an isotopic variant, a tautomer, or the like thereof. In various examples, a composition does not comprise guide RNA.

In various examples, a composition further comprises one or more cationic lipid(s). In various examples, a composition further comprises Lipofecatmine™ Transfection Agent (Invitrogen™) or the like. In various examples, a composition comprises a plurality of lipoplexes (e.g., formed by electrostatic interaction between anionically functionalized polypeptides and the cationic lipid(s)).

In various examples, a composition further comprises a combination of lipids (e.g., a combination of lipids that can form lipid nanoparticles (LNPs)). In various examples, a composition further comprises one or more ionizable lipid(s) (such as, for example, MC3 or the like), which may be pH-responsive cationic lipid(s) or the like), one or more zwitterionic lipid(s) (such as, for example, distearoylphosphatidylcholine (DSPC) or the like), cholesterol, one or more PEG functionalized lipid(s) (distearoyl-rac-glycerol-methoxypoly(ethylene) glycol (DSG-PEG) or the like), and optionally, a permanently cationically charged lipid (such as, for example, a cationic lipid comprising a permanently charged quaternary ammonium group (e.g., 1,2-dioleoyl-3-trimethylammonium-propane (DOTAP) or the like)) or the like). Suitable combinations of lipids (e.g., combinations of lipids which form lipid nanoparticles) are known in the art. In various examples, a composition comprises a plurality of lipid nanoparticles (e.g., formed by interaction between anionically functionalized polypeptides and the lipids). In various examples, the anionically functionalized polypeptide(s) are disposed partially or completely encapsulated by the lipid nanoparticle(s).

As used herein, unless otherwise indicated, the term “pharmaceutically acceptable” refers to those components and dosage forms that are, within the scope of sound medical judgment, suitable for use in contact with the tissues of humans or other animals without excessive toxicity, irritation, or other problem or complication, commensurate with a reasonable benefit/risk ratio.

Some non-limiting examples of materials which can be used as additional component(s) in a composition include sugars, such as, for example, lactose, glucose, sucrose, and the like; starches, such as, for example, corn starch, potato starch, and the like; cellulose, and its derivatives, such as, for example, sodium carboxymethyl cellulose, ethyl cellulose, cellulose acetate, and the like; powdered tragacanth; malt; gelatin; talc; excipients, such as, for example, cocoa butter, suppository waxes, and the like; oils, such as, for example, peanut oil, cottonseed oil, safflower oil, sesame oil, olive oil, corn oil, soybean oil, and the like; glycols, such as, for example, propylene glycol and the like; polyols, such as, for example, glycerin, sorbitol, mannitol, polyethylene glycol, and the like; esters, such as, for example, ethyl oleate, ethyl laurate, and the like; agar; buffering agents, such as, for example, magnesium hydroxide, aluminum hydroxide, and the like; alginic acid; pyrogen-free water; isotonic saline; Ringer's solution; ethyl alcohol; phosphate buffer solutions; and other non-toxic compatible substances employed in pharmaceutical formulations; and any combination thereof (See, e.g., Remington: The Science and Practice of Pharmacy (2012) 22nd Edition, Philadelphia, PA. Lippincott Williams & Wilkins).

In an aspect, the present disclosure provides bioconjugation reagents. In various examples, a bioconjugation reagent comprises a conjugation group, a cleavable group, optionally one or more linking group(s), and one or more anionic group(s). In various examples, a bioconjugation reagent is a means to render a polypeptide (e.g., a protein or a peptide) capable of intracellular transport (e.g., transport across the lipid bilayers of a cell or the like). In various examples, a bioconjugation group is suitable for use in a method of making an anionically functionalized polypeptide (e.g., an anionically functionalized protein or peptide) of the present disclosure. Non-limiting examples of bioconjugation reagents are described herein.

In various examples, bioconjugation reagent comprises the following structure: G′-X-L-(R)ₓ. G′ comprises (or is) a conjugation group; X comprises (or is) a cleavable group; L comprises (or is) a linking group; and R comprises (or is) an anionic group. In various examples, x is 1, 2, 3, 4, 5, or 6. In various examples, X, L, and R are as defined above with respect to the anionically functionalized polypeptide.

In various examples, a bioconjugation group comprises a conjugation group covalently bonded to a cleavable group, and a cleavable group covalently bonded to one or more anionic group(s). The covalent bonds may include one or more linking group(s) between, for example, a conjugation group and a cleavable group or between a cleavable group and one or more anionic group(s). In various examples, the cleavable group is β- to the conjugation group. As an illustrative example, a disulfide bond of a cleavable group is β- to a carbamate group of a conjugation group.

In various examples, a bioconjugation reagent comprises (or consists of) the following structure:

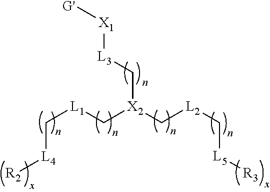

or a structural analog thereof. G′ comprises a conjugation group; X₁ comprises the cleavable group; X₂ is chosen from an N group, a CH group, a P group, a P═O group, and the like; L₁ and L₂ are independently chosen from —OC(O)—, —C(O)O—, —NHC(O)—, —C(O)NH—, —OC(O)NH—, —NHC(O)O—,

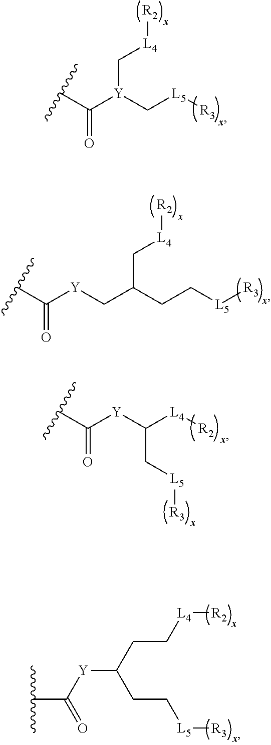

and structural analogs thereof, where Y is independently chosen from an NH group, an O group, an S group, a CH₂ group, an H group, a CH group, and the like; L₃ is chosen from —OC(O)—, —C(O)O—, —NHC(O)—, —C(O)NH—, —OC(O)NH—, —NHC(O)O—, and structural analogs thereof, L₄ and L₅ are independently optional and chosen from alkyl groups, alkenyl groups, and the like; and R₂ and R₃ are independently optional, wherein the bioconjugation reagent comprises at least one R₂ and/or R₃, and are chosen from —CO₂⁻, —CO₂H, —SO₃⁻, —SO₃H, —SO₂⁻, —PO₂H²⁻, —PO₃H⁻, —PO₄H²⁻, polymeric groups comprising one or more of the anionic groups(s), and structural analogs thereof. In various examples, n is independently at each occurrence 0, 1, 2, 3, 4, 5, or 6. In various examples, x is independently at each occurrence 1, 2, 3, 4, 5, or 6.

In various examples, a bioconjugation reagent comprises (or consists of) the following structure:

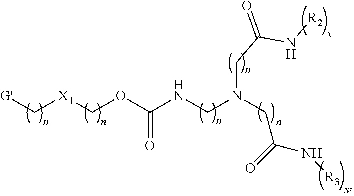

or a structural analog thereof.

In various examples, a bioconjugation reagent comprises (or consists of) the following structure:

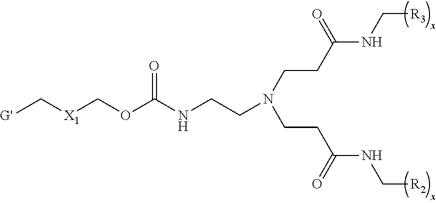

or a structural analog thereof.

In various examples, a bioconjugation reagent comprises (or consists of) the following structure:

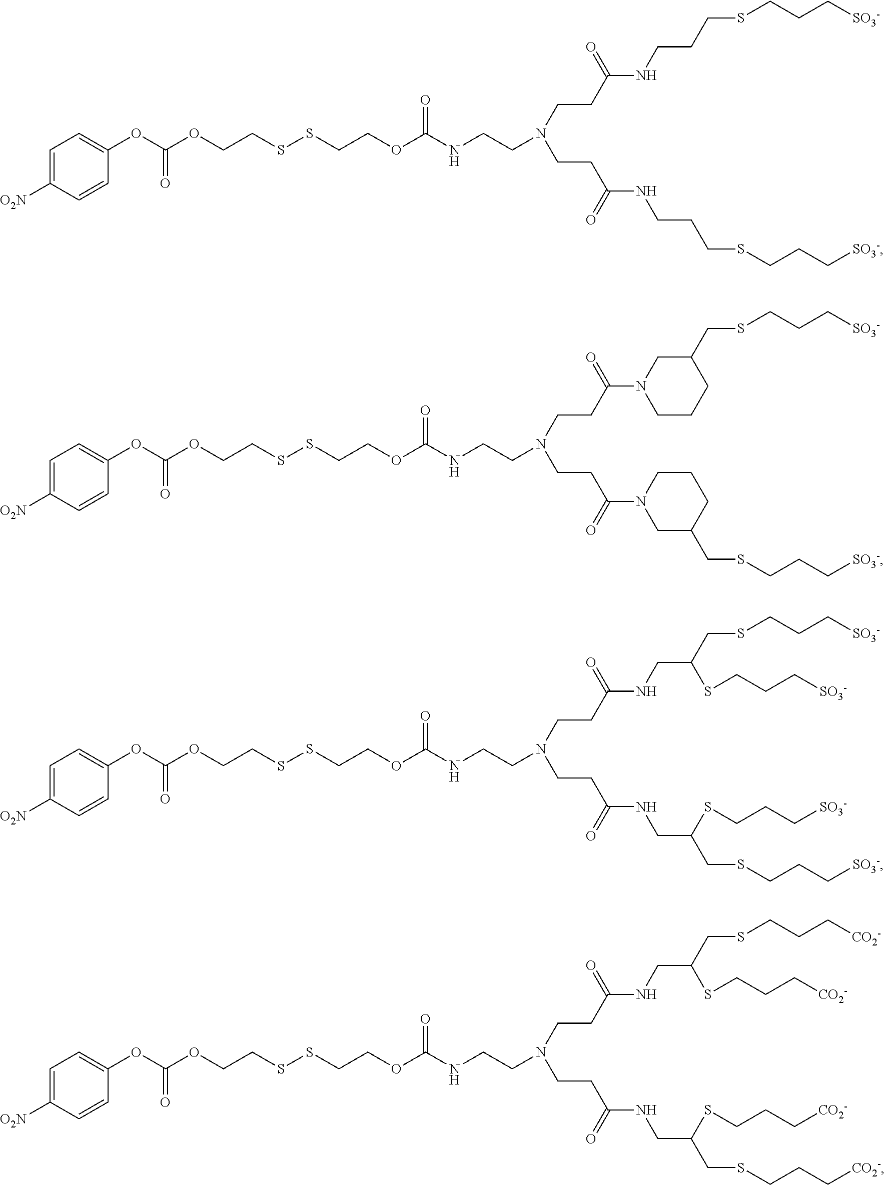

or a structural analog thereof or a pharmaceutically acceptable salt, a salt, a partial salt, a solvate, a polymorph, a prodrug, or the like thereof, or a stereoisomer or a mixture of stereoisomers, an isotopic variant, a tautomer, or the like thereof.

In various examples, a bioconjugation reagent comprises (or consists of) the structure:

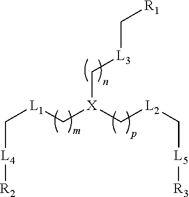

or a structural analog thereof. R₁ comprises (or is) a carbonate group, an ester group, or the like. In various examples, an ester group comprises (or is) an NHS ester group or a carbonate group comprises (or is) a nitrophenol carbonate group, a pentafluorophenyl carbonate group, a trifluorophenyl carbonate group, a hexafluoropropanol carbonate group, a trimethylaminophenyl carbonate group, or a structural analog thereof. n, m and p are independently 0, 1, 2, 3, 4, 5, or 6. L₁, L₂, and L₃, are independently chosen from —OC(O)—, —C(O)O—, —NHC(O)—, —C(O)NH—, —OC(O)NH—, —NHC(O)O—, and structural analogs thereof, and X is chosen from N, CH, P, P═O, and structural analogs thereof. In various examples, L₁ and L₂, are independently chosen from branch points, such as, for example:

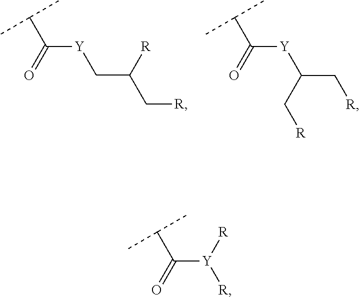

and structural analogs thereof, where R is —CH₂-L₄-R₂ or —CH₂-L₅-R₃. L₄ and L₅ are independently chosen from alkyl groups (such as, for example, C₁₋₄ alkyl groups, C₁₋₄ cycloalkyl groups, or the like) and alkenyl groups (such as, for example, C₁₋₄ alkenyl groups or the like), or the like. R₂ and R₃ are independently chosen from —CO₂⁻, —CO₂H, —SO₃⁻, —SO₃H, —SO₂⁻, —PO₂H²⁻, —PO₃H⁻, —PO₄H²⁻, polymeric groups comprising one or more of the anionic groups(s), and structural analogs thereof. Y is chosen from an NH group, an O group, an S group, a CH₂ group, an N group, a CH group, and structural analogs thereof.

In various examples, a bioconjugation reagent comprises (or consists of) the following structure:

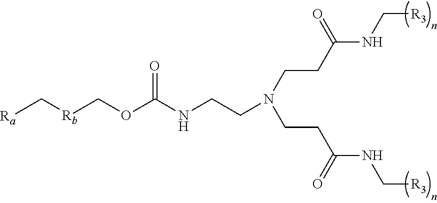

or a structural analog thereof. In various examples, Rₐ comprises (or is) conjugation group (e.g., a carbonate group, an ester group, or a structural analog thereof) and/or R_{b} comprises (or is) a cleavable group (e.g., comprises (or is) a stimuli-cleavable bond or the like) and/or R₃ independently comprises an anionic group. In various examples, an ester group comprises (or is) an NHS ester or a structural analog thereof. In various examples, a carbonate group comprises (or is) a nitrophenol carbonate, pentafluorophenyl carbonate, trifluorophenyl carbonate, hexafluoropropanol carbonate, a trimethylaminophenyl carbonate, or a structural analog thereof and/or a stimuli-cleavable bond comprises (or is) a redox-cleavable disulfide bond, a light-cleavable bond, an ROS-cleavable bond, a pH-cleavable bond, or the like and/or an anionic group comprises (or is) a CO₂⁻ group, a CO₂H group, a SO₃⁻ group, a SO₃H group, a SO₂⁻ group, a PO₂H²⁻ group, a PO₃H⁻ group, a PO₄H²⁻ group, a polymeric group comprising one or more of the anionic groups(s), or a structural analog thereof. In various examples, n is 1, 2, 3, 4, 5, or 6.

In various examples, a bioconjugation reagent comprises a conjugation group or the like. In various examples, a conjugation group comprises (or is) an N-hydroxysuccinimide ester group, a nitrophenol carbonate group, a pentafluorophenyl carbonate group, a trifluorophenyl carbonate group, a hexafluoropropanol carbonate group, a trimethylaminophenyl carbonate group, or the like. In various examples, a conjugation group has the following structure:

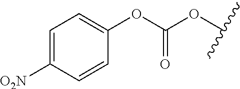 [SMILES: *OC(=O)Oc1ccc([N+](=O)[O-])cc1]

or a structural analog thereof.

In an aspect, the present disclosure provides methods of making anionically functionalized polypeptides. In various examples, a method is based on a reaction of a polypeptide (e.g., a protein or a peptide) with one or more bioconjugation reagent(s). In various examples, a method produces an anionically functionalized polypeptide of the present disclosure. Non-limiting examples of polypeptides and anionically functionalized polypeptides are described herein.

In various examples, a method of making an anionically functionalized polypeptide/polypeptides of the present disclosure comprises forming a reaction mixture comprising: one or more polypeptide(s) (non-anionically-functionalized polypeptide(s), such as, for example, modified polypeptide(s)) and one or more bioconjugation reagent(s); and holding the reaction mixture (e.g., for a time and/or at a temperature), where the anionically functionalized polypeptide (e.g., protein or peptide) is formed. In various examples, at least a portion or all of the anionically functionalized polypeptide is isolated. In various examples, the method does not comprise genetically encoding anionic polypeptides into a protein using naturally anionic protein complexes.

In various examples, a conjugation group of a bioconjugation reagent reacts with a sidechain of an amino acid residue of a polypeptide to form a conjugated group. In various examples, the X of a conjugated group

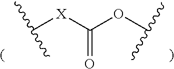

is derived from the sidechain of a polypeptide amino acid residue. As an illustrative example, a conjugated group comprises a carbamate, where X is NH, where the carbamate is formed from or derived from a carbonate of a conjugation group of the bioconjugation reagent (e.g., a conjugation group with a structure of

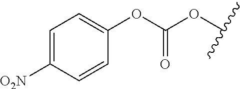 [SMILES: *OC(=O)Oc1ccc([N+](=O)[O-])cc1]

reacts with a polypeptide amino acid sidechain to form the carbamate having a structure of

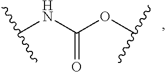 [SMILES: *NC(=O)O*]

where the NH is from the side chain of the amino acid residue).

The polypeptide is not particularly limited. In various examples, a polypeptide is a functional polypeptide or the like, or a fragment thereof. Non-limiting examples of polypeptides include enzymes, receptor ligands, transcriptional factors, growth factors, antibodies (or antigen-binding fragments thereof, such as, for example, single-chain antibody fragments, Fabs or the like, peptide or protein immunogens (e.g., that can be used for stimulating an immune response (e.g., a vaccine or the like) or the like), protein-based therapeutic agents (such as, for example, protein-based chemotherapeutic agents or the like), toxins, cytokines, hormones, fluorescent proteins, and the like, any fragments thereof, and any combination thereof. In various examples, a polypeptide does not comprise a protein genetically fused with the polypeptide.

In various examples, the polypeptide is any type of Clustered Regularly Interspaced Short Palindromic Repeats (CRISPR) enzyme or the like, including, but not limited to any Cas protein (such as, for example, single effector Cas proteins, including, but not limited to, those with enzymatic activity and those that are enzymatically dead, including, but not limited to, Cas9, Cas12a, and Cas13 and the like). In various examples, a polypeptide comprises a reverse transcriptase, which may be used, for example, in prime editing or the like. In various examples, the polypeptide comprises a cellular localization domain (e.g., so that the polypeptide formed from the anionically functionalized polypeptide can be trafficked to a cellular location, such as, for example, an organelle or the like), a nuclear localization signal, or the like, or both. In various examples, the anionically functionalized polypeptide is used for gene editing or otherwise modulating gene expression, or the like.

In various examples, a polypeptide is a modified polypeptide. In various examples, a modified polypeptide comprises a modification described herein. In various examples, an modified polypeptide comprises one or more cyclodextrin group(s), comprise one or more lipid(s), is acetylated at one or more modified polypeptide residue(s), is ubiquitinated at one or more modified polypeptide residue(s), is myristoylated at one or more modified polypeptide residue(s), is methylated at one or more modified polypeptide residue(s), is chemically modified at one or more modified polypeptide residue(s), or the like, or any combination thereof.

A polypeptide (such as, for example, a modified polypeptide or the like) can have various molecular weights. In various examples, a polypeptide has a molecular weight of 1 kilodalton (kDa) to 300 kDa, including all 0.1 kDa values and ranges therebetween (e.g., 10 kDa to 200 kDa or 15 kDa to 115 kDa).

A reaction mixture may comprise or more solvent(s). Non-limiting examples of suitable solvents are known in the art.

In various examples, at least a portion or all the polypeptide(s) is/are isolated. Suitable isolation methods are known in the art. In various examples, at least a portion or all the polypeptide(s) is/are isolated by filtration, centrifugation, precipitation, chromatography, or the like, or any combination thereof.

A reaction can be performed under various reaction conditions. A reaction can comprise one or more step(s) and each step can be performed under the same or different reaction conditions as other steps. A reaction can be carried out at various temperatures. In various examples, a reaction is carried out at about room temperature (e.g., from about 20° C. to about 30° C., including all 0.1° C. values and ranges therebetween), below room temperature, or above room temperature. A reaction can be carried out at various pressures. In various examples, a polymerization reaction is carried out at about atmospheric pressure (e.g., 1 standard atmosphere (atm) at sea level), at greater than atmospheric pressure, at below atmospheric pressure. A reaction can be carried out for various times. The reaction time can depend on factors such as, for example, temperature, pressure, mixing (e.g., stirring or the like), or the like, or any combination thereof. In various examples, reaction times range from about minutes (e.g., about 1 minute) to greater than about 24 hours, including all integer second values and ranges therebetween. In various examples, a method is carried out in air or an inert atmosphere. In various examples, a method is carried out in the presence of oxygen (which may be an oxygen atmosphere or the like). In various examples, a method is carried out in the absence of oxygen.

In an aspect, the present disclosure provides methods of using polypeptides of the present disclosure. In various examples, a method is a cell transfection method (e.g., in vitro, an ex vivo or an in vivo cell transfection method). Non-limiting examples of methods of using polypeptides of the present disclosure are described herein.

In various examples, the present disclosure provides a means for intracellular delivery of an anionically functionalized polypeptide or polypeptides. The intracellular delivery may be in vitro, ex vivo, or in vivo, or the like.

In various examples, one or more anionically functionalized polypeptide(s) and/or one or more composition(s) of the present disclosure are used in cell transfection methods. In various examples, a method of cell transfection comprises (or consist essentially of or consist of) contacting one or more cell(s) (e.g., cell population(s) or the like) with one or more anionically functionalized polypeptide(s) and/or one or more composition(s) to a subject. The method may be a an in vitro, an ex vivo or an in vivo cell transfection method.

In various examples, one or more anionically functionalized polypeptide(s) and/or one or more composition(s) of the present disclosure are used in treatment methods. In various examples, a method of treatment comprises (or consist essentially of or consist of) administration of one or more anionically functionalized polypeptide and/or one or more composition(s) to a subject.

In various examples, in an intracellular delivery method, a cell transfection method, or a treatment method, at least a portion of, substantially all, or all of the polypeptide(s) are delivered to a cell or cells and, after intracellular delivery, the native polypeptide(s) is/are formed in (released within) the cell (e.g., by reaction of the cleavable group of the anionically functionalized polypeptide(s)).

A method may treat a subject diagnosed with or in need of treatment for a disease, a disorder, or the like, or any combination thereof. In various examples, a method for treating a disease, disorder, or the like, or any combination thereof, comprises administering to a subject an amount of one or more anionically functionalized polypeptide(s) and/or one or more composition(s) of the present disclosure (one or more or all of which may be present as pharmaceutical composition), where one or more symptom(s), indication(s), or the like, or any combination thereof, of the subject is at least partially or completely alleviated. A method may treat a disease, a disorder, or the like, or any combination thereof that is treatable with an unfunctionalized (e.g., native, as synthesized, or the like) polypeptide.

A subject (e.g., a subject in need of treatment or the like) may be a human or other animal (which may be a non-human mammal). Non-limiting examples of non-human animals (which may be mammals) include cows, pigs, mice, rats, rabbits, cats, dogs, and other agricultural animals, pets (such as, for example, dogs, cats, and the like), service animals, and the like.

“Treating” or “treatment” of any disease or disorder refers, in various examples, to ameliorating (e.g., arresting, reversing, alleviating, or the like) the disease, disease state, condition, disorder, side effect, potential disease, potential disease state, potential condition, potential disorder, potential side effect, or the like, or a combination thereof, or reducing the manifestation, extent or severity of one or more clinical symptom(s) thereof, or the like. In various other examples, “treating” or “treatment” refers to ameliorating one or more physical parameter(s), which, independently, may or may not be discernible by the subject. In yet other examples, “treating” or “treatment” refers to modulating disease, disease state, condition, disorder, side effect, or the like, or a combination thereof, either physically, (e.g., stabilization of one or more discernible symptom(s), or the like), physiologically, (e.g., stabilization of one or more physical parameter, or the like), or both. In yet other examples, treating” or “treatment” relates to slowing the progression of the disease, disease state, condition, disorder, side effect, or the like, or a combination thereof. Treating may include administration of an effective amount of the composition(s).

As used herein, unless otherwise indicated, the term “effective amount” means that amount of the compound(s) and/or composition(s) that will elicit the biological or medical response of subject (or a tissue, system, or the like, thereof) that is being sought, for instance, by a researcher, clinician, or the like. An effective amount may be a therapeutically effective amount. The term “therapeutically effective amount” includes any amount which, as compared to a corresponding subject who has not received such amount, results in improved treatment, healing, prevention, or amelioration of a disease, disease state, condition, disorder, side effect, potential disease, potential disease state, potential condition, potential disorder, potential side effect, or the like, or a combination thereof, or a decrease in the rate of advancement of a disease, disease state, condition, disorder, potential disease, potential disease state, potential condition, potential disorder, potential side effect, or the like, or the like. The term also includes within its scope amounts effective to enhance normal physiological function.

An effective amount may result in prophylaxis. The term “prophylaxis” includes prevention and refers to a measure or procedure which is to prevent rather than cure or treat a disease. Preventing may refer to a reduction in risk of acquiring or developing a disease causing at least one clinical symptom of the disease not to develop in a subject that may be exposed to a disease causing agent or a subject predisposed to the disease in advance of disease outset.

A physician or veterinarian having ordinary skill in the art can readily determine and prescribe the effective amount of the compound(s) and/or composition(s) required. The selected dosage level can depend upon a variety of factors including, but not limited to, the activity of the particular composition employed, the time of administration, the rate of excretion or metabolism of the particular composition being employed, the rate and extent of absorption, the duration of the treatment, other drugs, compounds and/or materials used in combination with the particular composition employed, the age, sex, weight, condition, general health and prior medical history of the patient being treated, and like factors well known in the medical arts. For example, the physician or veterinarian could start doses of the composition employed at levels lower than that required in order to achieve the desired therapeutic effect and gradually increase the dosage until the desired effect is achieved.

In an aspect, the present disclosure provides kits. In various examples, a kit comprises one or more polypeptide(s), one or more bioconjugation reagent(s), one or more composition(s), or any combination thereof. Non-limiting examples of kits are described herein.

In various examples, a kit comprises one or more polypeptide(s), one or more bioconjugation reagent(s), one or more composition(s), or any combination thereof of the present disclosure. In various examples, a kit includes a closed or sealed package that contains the one or more polypeptide(s), one or more bioconjugation reagent(s), one or more composition(s), or any combination thereof. In various examples, the package comprises one or more closed or sealed vials, bottles, blister (bubble) packs, or any other suitable packaging for the sale, distribution, or use of the polypeptide(s), bioconjugation reagent(s), composition(s), or combination thereof. The printed material may include printed information. The printed information may be provided on a label, on a paper insert, printed on a packaging material, or the like. The printed information may include information that identifies the polypeptide(s), bioconjugation reagent(s), composition(s), or the combination thereof in the package, the amounts and types of other active and/or inactive ingredients in a composition comprising the bioconjugation reagent(s) or polypeptide(s), and instructions for using the polypeptide(s), one or more bioconjugation reagent(s), composition(s), or combination thereof.

In various examples, a kit comprises one or more anionically functionalized polypeptide(s) and/or composition(s) (e.g., one or more pharmaceutical composition(s)) of the present disclosure). In various examples, a kit includes a closed or sealed package that contains the one or more one or more anionically functionalized polypeptide(s) and/or composition(s). In various examples, the package comprises one or more closed or sealed vials, bottles, blister (bubble) packs, or any other suitable packaging for the sale, distribution, or use of the one or more compound(s) and/or composition(s). The printed material may include printed information. The printed information may be provided on a label, on a paper insert, printed on a packaging material, or the like. The printed information may include information that identifies the compound(s) in the package, the amounts and types of other active and/or inactive ingredients in the composition(s), and instructions for taking the polypeptide(s) and/or composition(s). The instructions may include information, such as, for example, the number of doses to take over a given period of time, and/or information directed to a pharmacist and/or another health care provider, such as, for example, a physician or the like, or a patient. The printed material may include an indication or indications that the one or more anionically functionalized polypeptide(s) and/or composition(s) and/or any other agent provided therein is for treatment of a subject. In various examples, the kit includes a label describing the contents of the kit and providing indications and/or instructions regarding use of the contents of the kit to treat a subject.

The following Statements provide examples of bioconjugation reagents, polypeptides, compositions, methods of making a polypeptide or polypeptides, and uses of the polypeptides (or compositions) of the present disclosure:

Statement 1. A bioconjugation reagent (such as, for example, a bioconjugation reagent described herein) comprising (or consisting essentially of or consisting of): a conjugation group (which may be an activated carbonate group, such as, for example, a p-nitrophenyl carbonate group or the like); a cleavable group (which may be a thiol group or the like); optionally one or more linking group(s) ((which may independently be (or comprise) one or more alkyl group(s)), and one or more anionic group(s) (which independently may be a protonated anionic, a deprotonated anionic group, or an anionic group salt) (which may independently be a sulfonic acid group, a sulfonate group or the like).
Statement 2. A bioconjugation reagent according to Statement 1, wherein the bioconjugation reagent comprises (or has) the following structure:

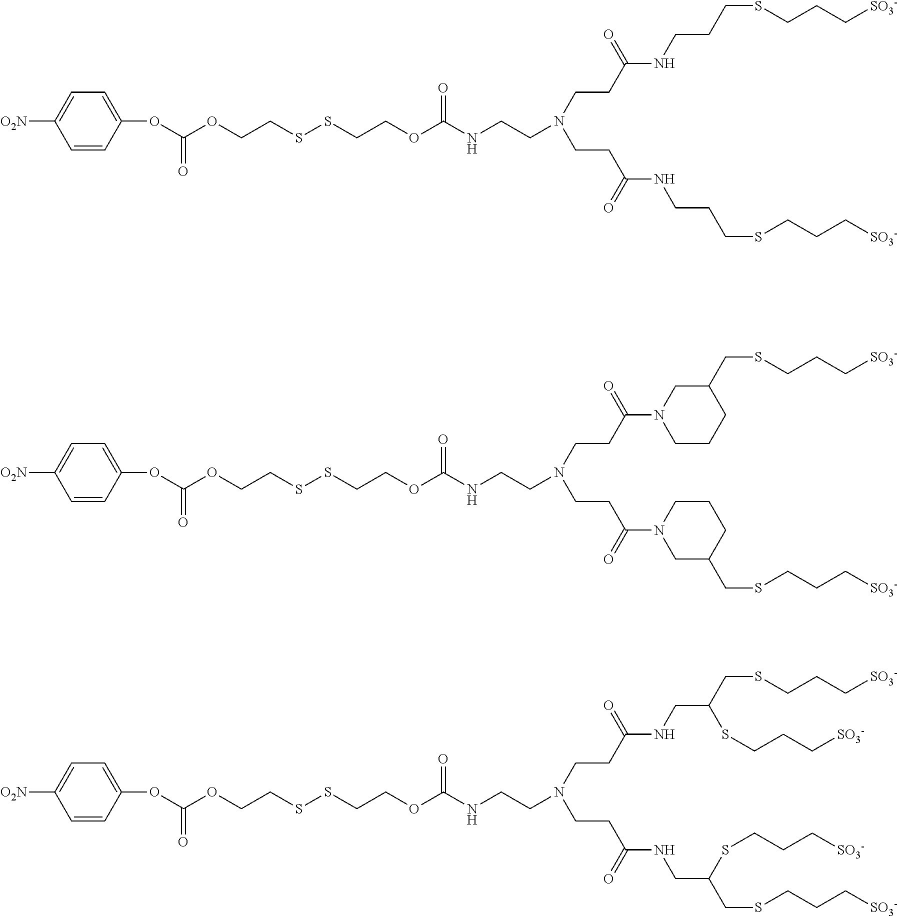

or the like, or a structural analog thereof wherein any one or more or all of the alkyl group(s) are independently chosen from C₁, C₂, C₃, C₄, C₅, C₆, C₇, C₈ alkyl groups and the like.
Statement 3. A polypeptide (e.g., a protein, a peptide, or the like) (such as, for example, a bioconjugation reagent described herein) comprising one or more functional groups (which may be a structural analog or formed from a bioconjugation reagent of Statement 1 or Statement 2) comprising: a conjugated group (e.g., a structural analog of or formed from a conjugation group of a bioconjugation reagent of Statement 1 or 2) (which may be a carbamate group or the like); a cleavable group (which may be a thiol group or the like); optionally one or more linking group(s) (which may independently be (or comprise) one or more alkyl group(s)); and one or more anionic group(s) (which independently may be a protonated anionic, a deprotonated anionic group, or an anionic group salt) (which may independently be a sulfonic acid group, a sulfonate group, or the like), or a pharmaceutically acceptable salt, a salt, a partial salt, a solvate, a polymorph, or a prodrug thereof, or an isotopic variant thereof, wherein the anionic groups are covalently bonded to the polypeptide (e.g., via a lysine group of the polypeptide or the like).
Statement 4. A composition (e.g., a pharmaceutical composition or the like) comprising one or more polypeptide(s) of Statement 3.
Statement 5. A composition according to Statement 4, wherein the composition further comprises one or more cationic lipid reagent(s).
Statement 6. A composition according to Statement 4 or 5, wherein the composition further comprises one or more nanoparticles (such as, for example, lipid nanoparticles or the like) and the polypeptide(s) are disposed (e.g., sequestered, at least partially or completely encapsulated, or the like) by or within the nanoparticle(s).
Statement 7. A composition according to any one of Statements 3-6, wherein the composition is a pharmaceutical composition, and the composition further comprises one or more pharmaceutically acceptable excipient(s).
Statement 8. A method of making a polypeptide/polypeptides (e.g., a protein(s) or peptide(s)) of the present disclosure (e.g., a polypeptide(s) (e.g., protein(s) or peptide(s)) of any one of Statement 3 or the like), the method comprising: forming a reaction mixture comprising: one or more bioconjugation reagent(s) (e.g., bioconjugation reagent(s) of Statement 1 or 2, or the like), and one or more polypeptide(s) (e.g., protein(s), peptide(s), or the like, or any combination thereof) (which may be referred to as native polypeptide(s)); and holding the reaction mixture (e.g., for a time and at a temperature), wherein the polypeptide/polypeptides is/are formed.
Statement 9. A method according to Statement 8, wherein the polypeptide(s) is/are chosen from antibodies, cytokines, hormones, any fragments thereof, and the like, and any combination thereof.
Statement 10. A method according to any of Statements 8 or 9, wherein the reaction mixture further comprises one or more solvent(s).
Statement 11. A method of treating a subject diagnosed with or is in need of treatment, the method comprising administering to a subject an effective amount of one or more polypeptide(s) of Statement 3 (which may be included in a composition according to any one of Statements 4-7),
wherein one or more symptom(s) and/or indication(s) of the subject is at least partially alleviated.

The steps of the methods described in the various embodiments and examples disclosed herein are sufficient carry out the methods of the present disclosure. Thus, in an embodiment, a method consists essentially of a combination of the steps of the methods disclosed herein. In another embodiment, a method consists of such steps.

The following example is presented to illustrate the present disclosure. It is not intended to be limiting in any manner.

### Example

This example describes anionically functionalized polypeptides of the present disclosure, methods of making same, and uses thereof.

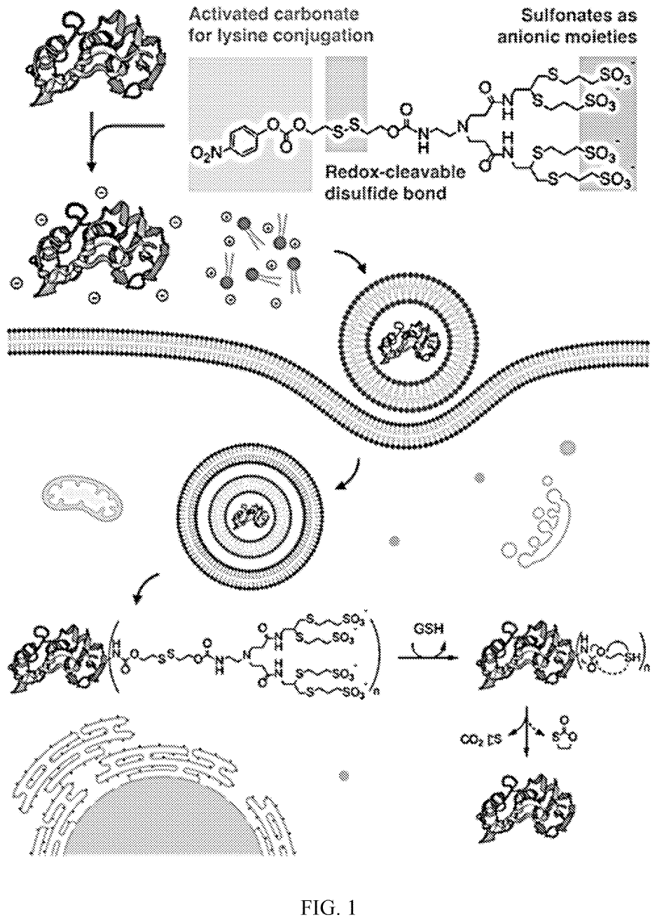

Bioreversible anionic cloaking enables intracellular protein delivery with ionizable lipid nanoparticles. We explored a reversible bioconjugation strategy that can endow proteins with an anionic “cloak” to facilitate electrostatic complexation with cationic lipids for intracellular delivery (FIG. 1). This is achieved through lysine-reactive compounds containing anionic sulfonate groups that can efficiently remodel the surface charge of any given protein cargo. Further, by utilizing self-immolative disulfide chemistry, the compounds can be cleaved from the delivered proteins within the reducing environment of the cytosol, offering a traceless method of protein delivery. We established this novel delivery approach and showcase its utility for the functional delivery of a variety of protein cargos, including a therapeutic enzyme and a full-length antibody, both in vitro and in vivo.

Results. Anionic bioconjugation enables intracellular protein delivery with commercial cationic lipid reagents. We reasoned that the formation of an effective anionic cloak would require global surface charge remodeling to endow sufficient anionic character to a cargo protein. To test this notion, we envisaged the use of activated carbonate compounds to chemoselectively attach sulfonate groups to surface-exposed lysine residues on proteins of interest. This bioconjugation-based approach allows for global, nonspecific charge reversal of positively charged lysine residues to negatively charged sulfonate groups via carbamate formation. Charge modification with sulfonates in particular would enable the formation of a strong anionic cloak due to their exceptionally low pKₐ (pKₐ≈−7). Additionally, incorporating a disulfide bond β- to the carbamate attachment enables redox-mediated cleavage and self-immolation to tracelessly regenerate the native protein within the reducing environment of the cytosol.

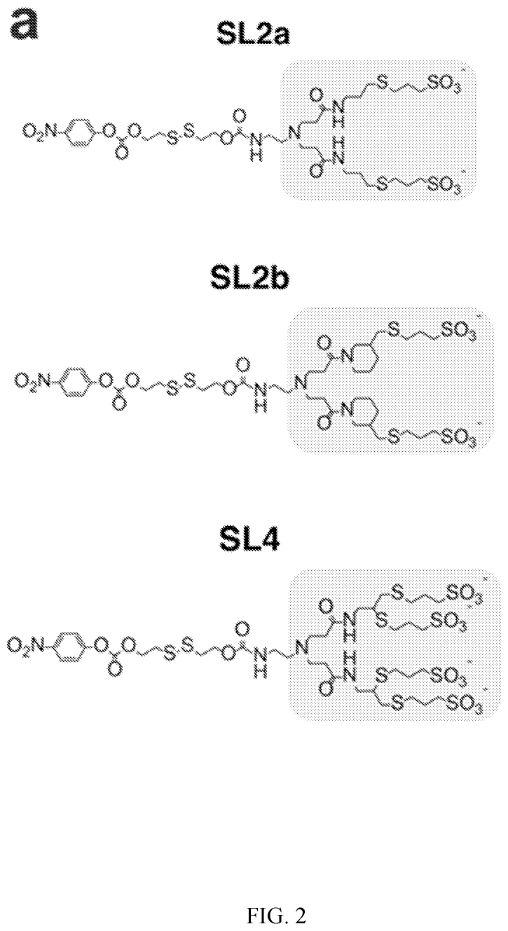

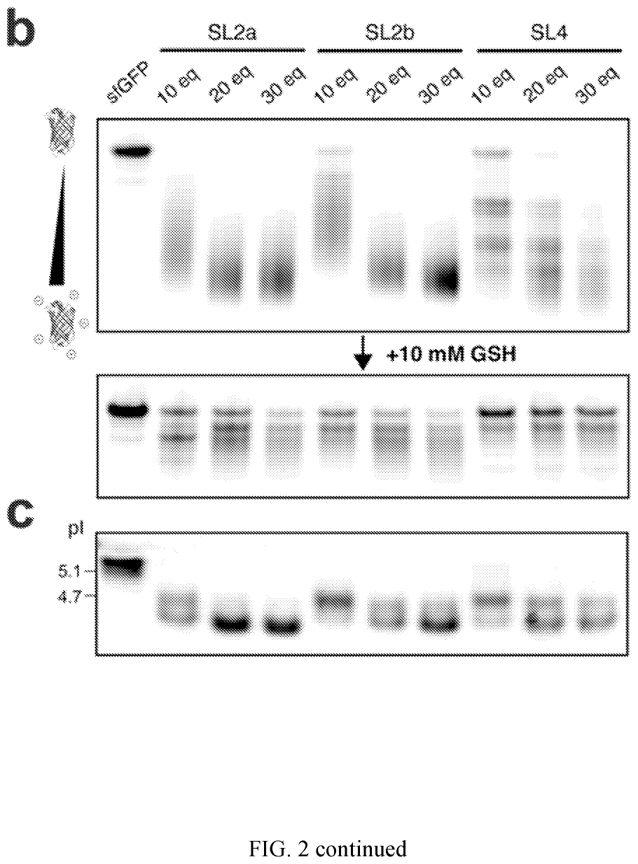

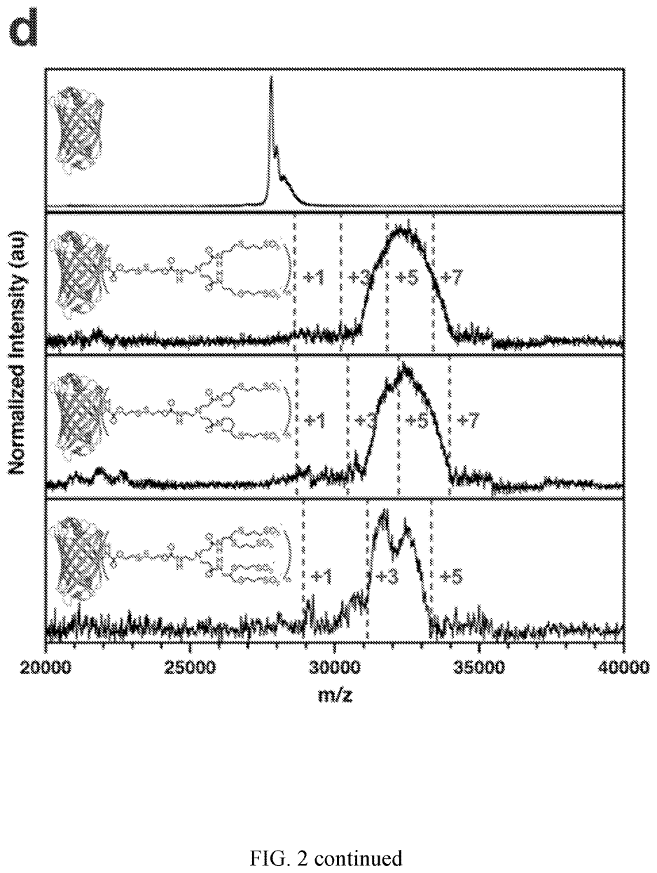

To validate this strategy, we synthesized a series of three sulfonated p-nitrophenyl carbonate compounds containing disulfide linkers (FIG. 2A) and employed superfolder green fluorescent protein (sfGFP) as a model protein to study the conjugation and delivery process. Successful anionic protein modification was confirmed by polyacrylamide gel electrophoresis run under native conditions (FIG. 2B). The addition of increasing molar equivalents of the three sulfonated compounds resulted in increasingly faster migration of the modified sfGFP through the gel. Given that the positive potential is oriented at the bottom of the gel, we attributed the faster migration of proteins to successful anionic surface charge modification. It is worth noting that sfGFP retains its intrinsic fluorescence upon chemical modification, as evidenced by the in-gel fluorescence images. Furthermore, complete protein modification occurred when sfGFP was reacted with 30 molar equivalents of each of the sulfonated compounds. Incubation of the modified sfGFP samples with 10 mM GSH (corresponding to the approximate concentration in a reducing cytosolic environment) resulted in convergence of the bands towards the unmodified sfGFP band, demonstrating successful disulfide cleavage and traceless recovery of the native protein. Anionic modification of sfGFP was further resolved through isoelectric focusing (IEF), which clearly showed a reduction in sfGFP isoelectric point (pI) to below 4.7 upon conjugation to the sulfonated compounds (FIG. 2C). Interestingly, the pI of sfGFP at full modification was approximately the same for all three sulfonated compounds, despite the fact that the compounds vary in overall hydrophobicity and valency of sulfonate groups. From MALDI-TOF-MS analysis, shifts in mass peaks arising from lysine-attached adducts revealed an average degree of conjugation ranging from 3 to 5 for the fully modified proteins (FIG. 2D). The resulting bioconjugation thus corresponds to an estimated decrease in the theoretical net charge of sfGFP from approximately −2 to a range between −11 to −27 under physiological conditions.

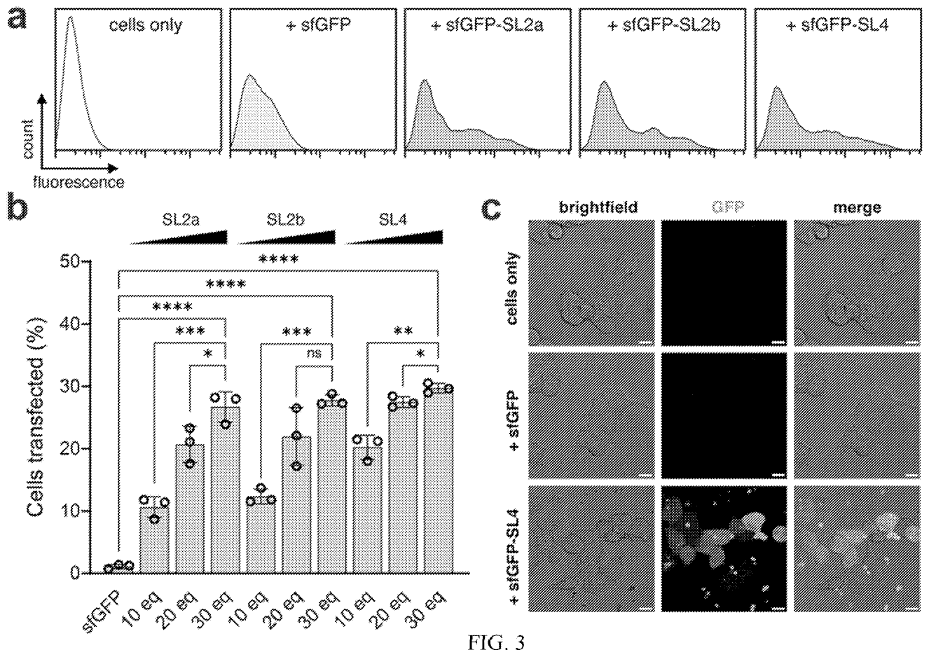

Having established efficient anionic bioconjugation, we next investigated the delivery of anionically-cloaked sfGFP into cells using Lipofectamine 2000 (LF2K), a commercial cationic lipid reagent routinely employed for in vitro transfection of nucleic acids. Flow cytometry experiments with 500 nM of anionically-cloaked sfGFP complexed with LF2K revealed elevated intracellular fluorescence in HEK293T cells (FIG. 3A), indicative of successful protein internalization. Conversely, cells treated with native, unmodified sfGFP complexed with LF2K exhibited no measurable delivery. Furthermore, fluorescent signals in cells were observed with sfGFP concentrations as low as 50 nM (FIG. 8). Efficiency of LF2K-mediated sfGFP delivery (quantified as percent GFP-positive cells) into cells increased with increasing amounts of sulfonate modification (FIG. 3B), suggesting that the degree of anionic protein modification may correlate with efficiency of electrostatic complexation with cationic lipids and, ultimately, delivery efficiency. However, maximal delivery efficiency with LF2K was capped at 30% for sfGFP that was reacted with 30 molar equivalents of all three sulfonated compounds. Confocal microscopy images corroborated the flow cytometry results and confirmed that protein internalization within cells only occurred when LF2K was complexed with anionically-cloaked sfGFP (FIG. 3C). Taken together, these results demonstrate that a ˜30 kDa globular protein can undergo anionic surface charge remodeling to enable electrostatic complexation and intracellular delivery with off-the-shelf cationic lipid reagents.

LNPs formulated with anionically-cloaked sfGFP results in robust protein internalization. Having established proof-of-principle of our protein delivery approach, we next sought to adapt our strategy for therapeutically relevant applications by utilizing clinically validated LNP formulations. Traditional LNP formulations consist of four lipid components—“ionizable” tertiary-amine containing lipids, zwitterionic phospholipids, cholesterol, and poly(ethylene) glycol (PEGylated) lipids—that are mixed at precise molar ratios to give rise to structured, homogenous nanoparticles. Essential to LNP formation with nucleic acids is the charge state of the ionizable lipid, which is modulated by the pH of the formulation mixture. In particular, ionizable lipids with pKₐ≈6.5 are able to (i) form electrostatic complexes with nucleic acids in acidic environments (e.g., buffers at pH 3), wherein the tertiary amines are protonated, and (ii) transition to an uncharged state at the physiological pH of 7.4. This feature is advantageous for minimizing off-target cytotoxicity during circulation.

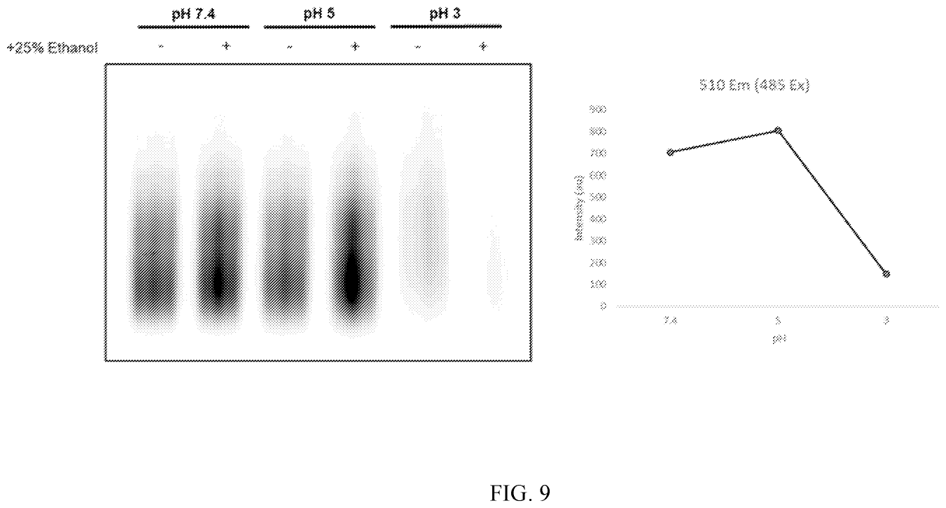

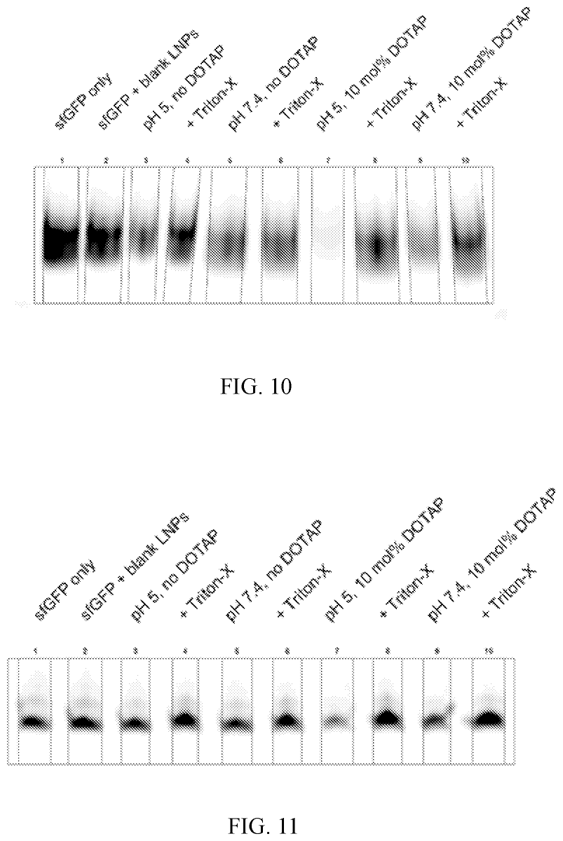

While traditional LNP formulations involve rapid mixing of an ethanolic lipid solution with an acidic aqueous buffer containing the nucleic acid, we were hesitant to adopt such a harsh method for protein cargos out of concern that it could induce unintended disruption of protein structure and function during the formulation process. As an example, sfGFP fluorescence was quenched when the protein was placed in citrate buffer at pH 3 but retained its fluorescence at higher pH ranges (FIG. 9). Shifting to higher formulation pH, however, would result in decreased populations of protonated ionizable lipids. To balance these competing effects, we hypothesized that the introduction of an auxiliary cationic lipid to the conventional four component LNP system would facilitate the electrostatic-driven assembly of LNPs with anionically-cloaked proteins in protein-friendly neutral pH buffers.

We chose to formulate LNPs with anionically-cloaked sfGFP (modified with 30 molar equivalents of SL4) using the “gold standard” ionizable lipid DLin-MC3-DMA (MC3) utilized in the FDA-approved siRNA-based drug Onpattro. LNPs of varying lipid amounts (2-10 wt/wt, MC3/sfGFP) were formed using a traditional four component system comprised of MC3, distearoylphosphatidylcholine (DSPC), cholesterol, and distearoyl-rac-glycerol-methoxypoly(ethylene) glycol (DSG-PEG) (50/10/38.5/1.5 mol/mol) along with a formulation supplemented with 10 mol % of 1,2-dioleoyl-3-trimethylammonium-propane (DOTAP), a cationic lipid comprising a permanently charged quaternary ammonium. Dynamic light scattering measurements (DLS) revealed successful formation of nanoparticles ranging between 200-300 nm in size with low polydispersity across all formulations made at both pH 5 and pH 7.4 (Table 1). Zeta potential measurements of the nanoparticles ranged from 0 and −5, signifying their overall neutral surface charge (Table 1). Interestingly, encapsulation efficiency of sulfonate-cloaked sfGFP was markedly higher when formulated with LNPs supplemented with DOTAP in both pH 5 and pH 7.4 buffers, suggesting that DOTAP may play a crucial role in increasing protein encapsulation with cationic lipids (Table 2).

TABLE 1
Size (z-avg), PDI, surface zeta potential of MC3 LNPs formulated with
sfGFP-SL4 (modified with 30 molar eq.). LNPs were formulated in pH 5
and pH 7.4 buffers and supplemented with or without 10 mol % DOTAP.

| | Z- | | | | Zeta | |
|---|---|---|---|---|---|---|
| | Average | Std | | Std | Potential | Std |
| | (nm) | Dev | PDI | Dev | (mV) | Dev |
| 2 wt/wt MC3 (pH 5) | 273.7 | 4.2 | 0.114 | 0.015 | −0.48 | 0.26 |
| 5 wt/wt MC3 (pH 5) | 273.8 | 7.4 | 0.132 | 0.013 | −1.04 | 1.16 |
| 10 wt/wt MC3 (pH 5) | 247.7 | 3.3 | 0.135 | 0.019 | −1.43 | 0.49 |
| 2 wt/wt MC3 + 10 mol % DOTAP (pH 5) | 290.6 | 3.0 | 0.086 | 0.014 | −0.45 | 0.96 |
| 5 wt/wt MC3 + 10 mol % DOTAP (pH 5) | 354.0 | 6.9 | 0.175 | 0.012 | −3.52 | 1.16 |
| 10 wt/wt MC3 + 10 mol % DOTAP (pH 5) | 294.6 | 7.1 | 0.110 | 0.024 | −2.56 | 0.42 |
| 2 wt/wt MC3 (pH 7.4) | 141.7 | 4.0 | 0.140 | 0.013 | −3.96 | 0.28 |
| 5 wt/wt MC3 (pH 7.4) | 181.0 | 2.8 | 0.191 | 0.020 | −3.43 | 0.93 |
| 10 wt/wt MC3 (pH 7.4) | 182.5 | 3.2 | 0.187 | 0.015 | −2.13 | 0.29 |
| 2 wt/wt MC3 + 10 mol % DOTAP (pH 7.4) | 242.9 | 3.2 | 0.129 | 0.027 | 0.42 | 0.81 |
| 5 wt/wt MC3 + 10 mol % DOTAP (pH 7.4) | 239.2 | 4.7 | 0.082 | 0.014 | −1.28 | 0.23 |
| 10 wt/wt MC3 + 10 mol % DOTAP (pH 7.4) | 211.3 | 5.5 | 0.121 | 0.016 | −5.41 | 1.46 |

TABLE 2
Encapsulation efficiency of sfGFP-SL4 in MC3 LNPs as calculated
from gel densitometry (n = 3). Percent encapsulation was
calculated by normalizing signal from LNP
samples with signal from LNP samples treated with Triton-X.

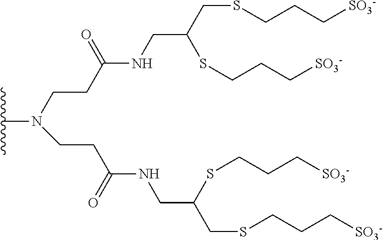

| | Encapsulation (%) | Std Dev |
|---|---|---|
| pH 5, no DOTAP | 25.76 | 10.46 |
| pH 7.4, no DOTAP | 9.18 | 2.61 |
| pH 5, 10 mol % DOTAP | 73.36 | 13.11 |
| pH 7.4, 10 mol % DOTAP | 40.04 | 11.15 |

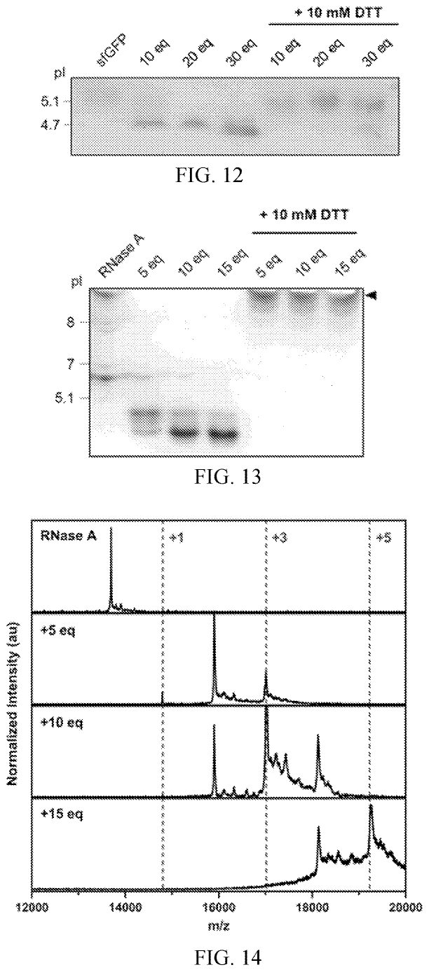

We next examined the effect of charge type on the anionic-cloaking mechanism by modifying sfGFP with p-nitrophenyl carbonate compounds containing carboxylate groups. In principle, surface charge modification with carboxylates, which possess a much higher pKₐ (pKₐ≈5) compared to sulfonates, would result in a weaker anionic cloak and, subsequently, less efficient complexation with ionizable lipids. This weaker anionic cloak was indeed evidenced by a reduced shift in the pI of sfGFP when it was cloaked with 30 molar equivalents of the carboxylated compound, CL4, compared to that of the sulfonated compound, SL4 (FIG. 12). As a result, encapsulation efficiency of CL4-cloaked sfGFP (sfGFP-CL4) within LNPs was notably reduced compared to SL4-cloaked sfGFP (sfGFP-SL4) (Table 3). Consistent with the previously observed trend, supplementing the LNP formulation with DOTAP, both at pH 5 and pH 7.4, led to improved encapsulation efficiency for sfGFP-CL4 (Table 3). This result further emphasizes the vital contribution of DOTAP in enhancing protein encapsulation with cationic lipids.

TABLE 3
Encapsulation efficiency of sfGFP-SL4 in MC3 LNPs as calculated
from gel densitometry (n = 3). Percent encapsulation was calculated by
normalizing LNP samples with LNP samples treated with Triton-X.

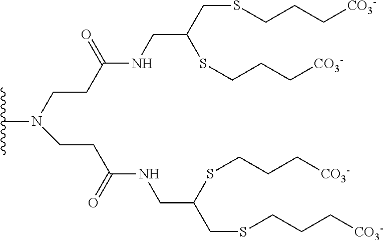

| | Encapsulation (%) | Std Dev |
|---|---|---|
| pH 5, no DOTAP | 13.43 | 3.64 |
| pH 7.4, no DOTAP | 10.30 | 7.40 |
| pH 5, 10 mol % DOTAP | 31.46 | 13.73 |
| pH 7.4, 10 mol % DOTAP | 30.70 | 5.89 |

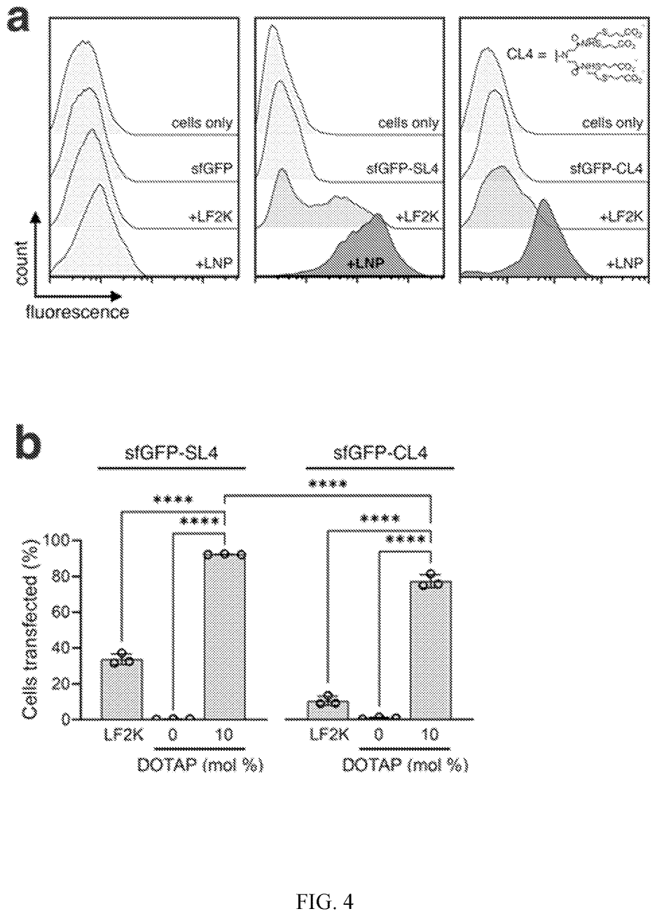

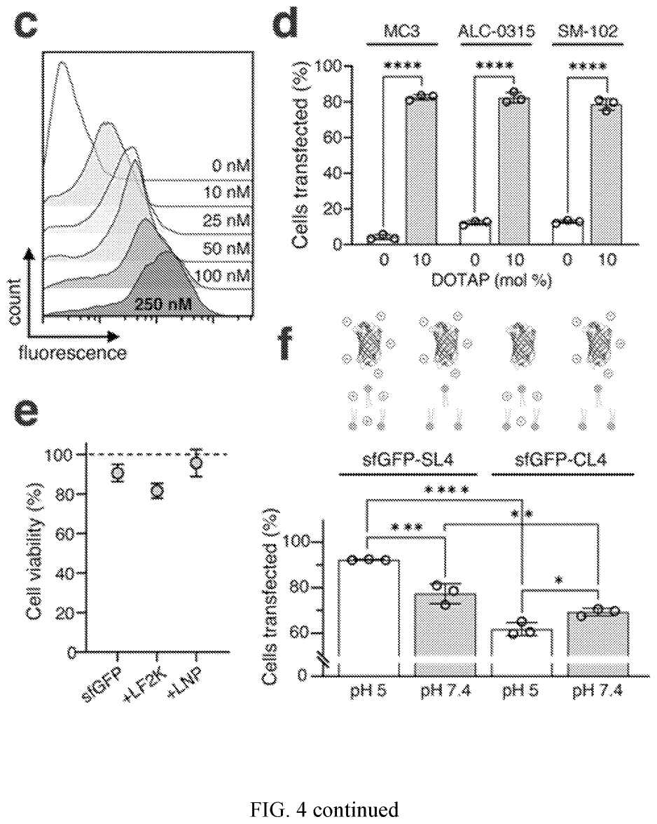

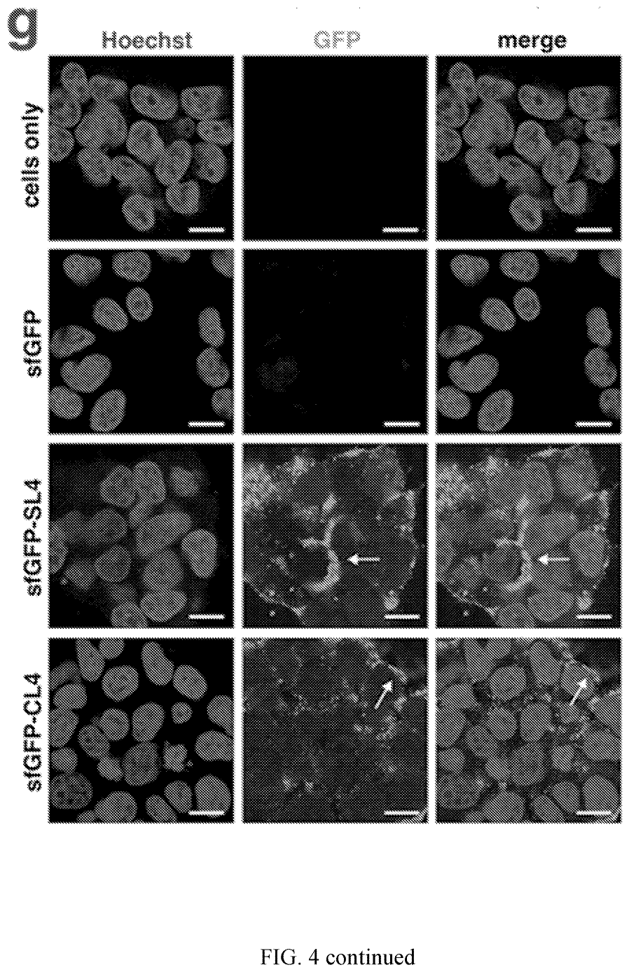

Subsequently, we examined whether the formulated LNPs were capable of transporting sfGFP into cells. Following transfection of 250 nM sfGFP into HEK293T cells, there was little evidence of protein delivery for MC3 LNPs formulated with unmodified sfGFP or LNPs formulated using the traditional four component lipid system. Strikingly, however, we observed a pronounced shift in intracellular fluorescence for anionically-cloaked sfGFP complexed with LNPs that were supplemented with 10 mol % DOTAP (FIG. 4A). The extent of protein delivery was particularly impressive when analyzing the delivery efficiencies, with nearly 90% of cells transfected with MC3 LNPs formulated with sfGFP-SL4 and an attenuated, yet robust, delivery efficiency of ˜70% for sfGFP-CL4 (FIG. 4B). Intracellular fluorescence was discernable in a dose-dependent manner, ranging from concentrations of sfGFP-SL4 at 250 nM down to 10 nM (FIG. 4C). Delivery of sfGFP-SL4 was also achieved using formulations of ALC-0315 and SM-102—the ionizable lipids used in the SARS-CoV-2 mRNA vaccines from Pfizer/BioNTech and Moderna, respectively—but only with formulations supplemented with DOTAP (FIG. 4D). These results illustrate the adaptability of the anionic cloaking strategy across various LNPs comprised of different ionizable lipid architectures but with the caveat that supplementation with an additional cationic lipid is necessary for LNP-mediated protein delivery. Furthermore, LNP-mediated protein delivery is nontoxic, with most cells exhibiting viabilities above 90% following treatment (FIG. 4E). This promising biocompatibility bodes well for potential in vivo applications.

Comparing delivery between sulfonate versus carboxylate-cloaked sfGFP, it was evident that surface charge modification with sulfonates led to enhanced delivery using both LF2K and MC3 LNPs (FIG. 4B), owing to the increased anionic character of the modified proteins. Specifically, delivery was higher for MC3 LNPs formulated with sfGFP-SL4 at both pH 5 and pH 7.4 of mixing compared to those formulated with sfGFP-CL4 (FIG. 4F). Interestingly, the highest protein delivery was achieved at pH 5 of mixing with sfGFP-SL4, whereas delivery of sfGFP-CL4 appeared roughly similar at both pH 5 and pH 7.4. The trends in delivery efficiency tended to correlate with sfGFP encapsulation efficiency of formulated LNPs, with sfGFP-SL4 achieving >70% encapsulation efficiency at pH 5 and ˜40% at pH 7.4, whereas encapsulation efficiency of sfGFP-CL4 was ˜30% when formulated with LNPs in both pH 5 and pH 7.4 buffers (Table 2 and Table 3).

Based on these data, and without intending to be bound to any specific theory, we propose a molecular-level mechanism as follows. At pH 5, sulfonate-cloaked proteins are predominantly anionic (since the pKₐ of sulfonates is less than 5), while ionizable lipids remain predominantly protonated (MC3 pKₐ≈6.5). This favors maximum electrostatic interactions between protein and lipid, resulting in maximal encapsulation and delivery. Shifting the pH of mixing to 7.4 maintains anionic charge of the sulfonate-cloaked proteins but induces deprotonation of the ionizable lipids (solution pH>pKₐ of MC3), which diminishes electrostatic interactions and reduces delivery efficiency. For carboxylate-cloaked proteins at pH 5, the proteins are relatively neutral (pKₐ of carboxylates≈5) while ionizable lipids remain protonated. At higher mixing pH, the carboxylate-cloaked proteins gain anionic character while ionizable lipids deprotonate. Thus, neither mixing pH provides an optimal environment for electrostatic interactions to occur between protein and lipid, resulting in reduced delivery efficiencies of carboxylate-cloaked sfGFP. The differences in delivery between the two anionic cloaks is supported by confocal microscopy images, where a more prominent GFP signal was observed in cells treated with sfGFP-SL4 compared to sfGFP-CL4 (FIG. 4G).

In summary, these findings illustrate three key points of our delivery strategy: 1) anionic cloaking of sfGFP is necessary for protein delivery with LNPs, 2) successful protein internalization with LNPs requires the use of modified formulations supplemented with permanently cationic lipids, and 3) modification of protein with anionic groups of different pKₐ's impacts LNP encapsulation and delivery efficiency.

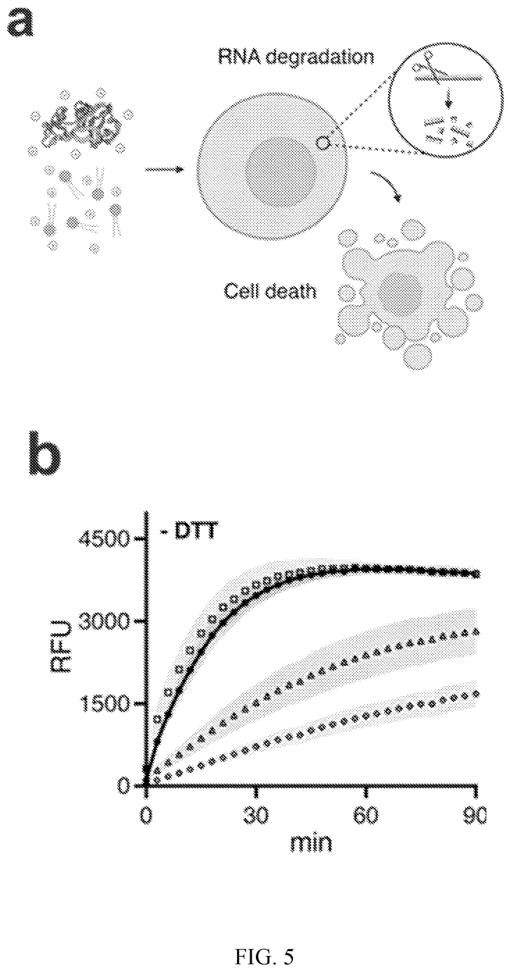

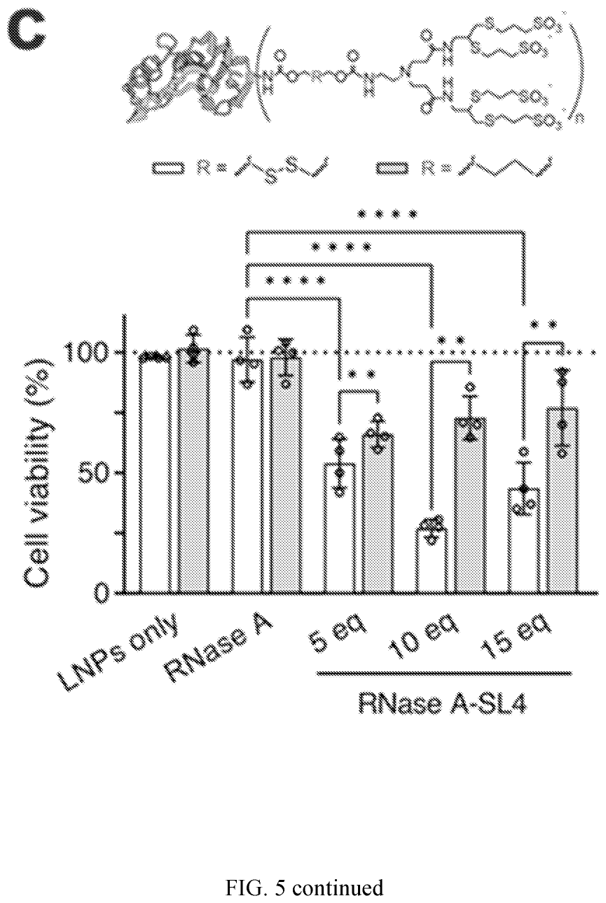

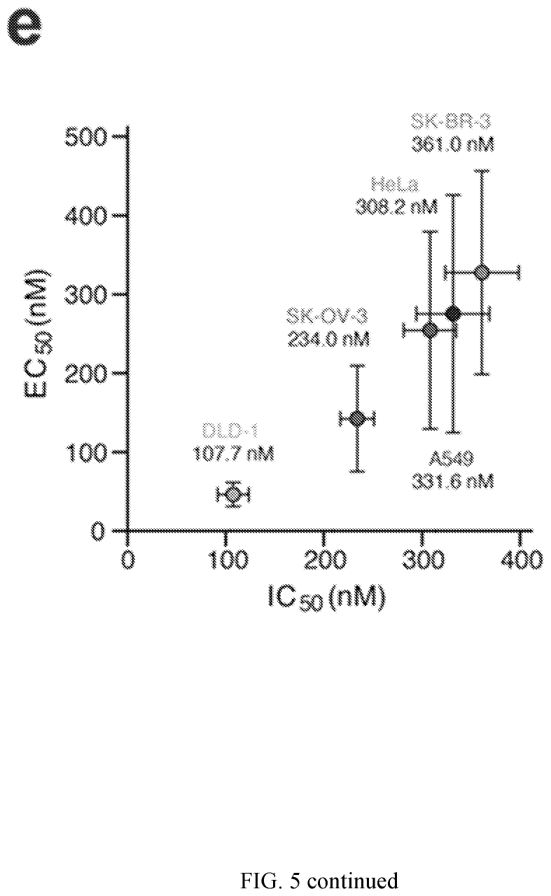

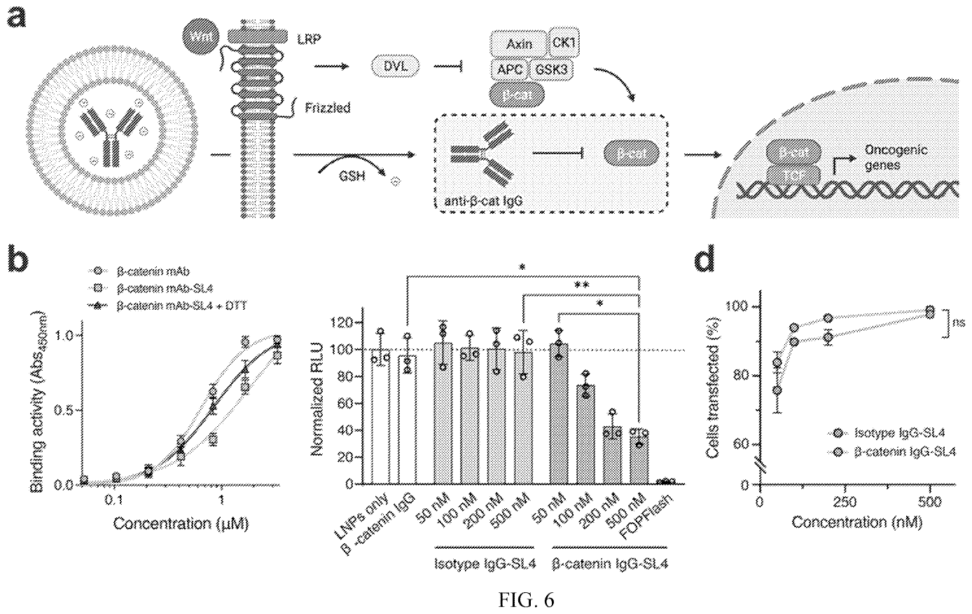

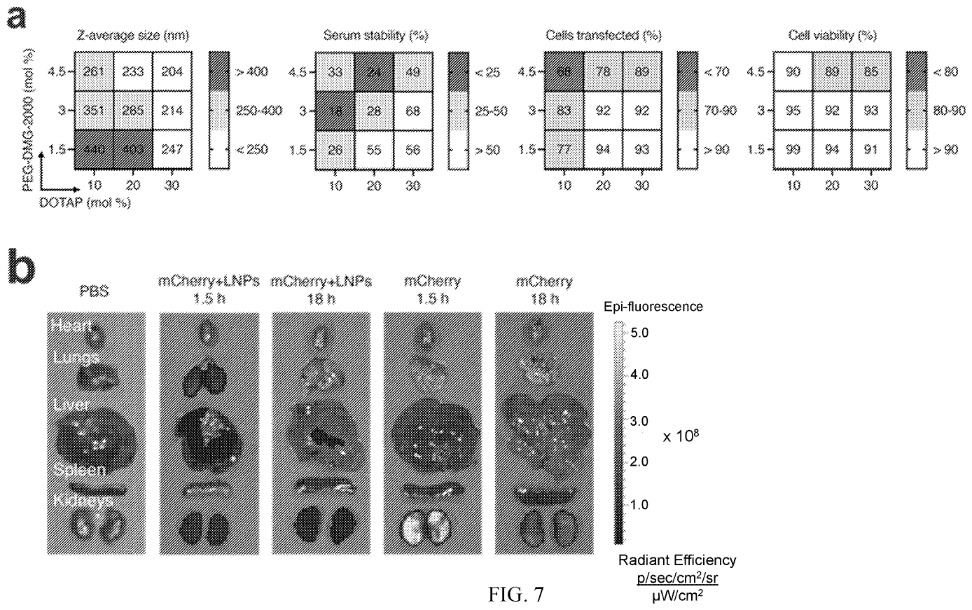

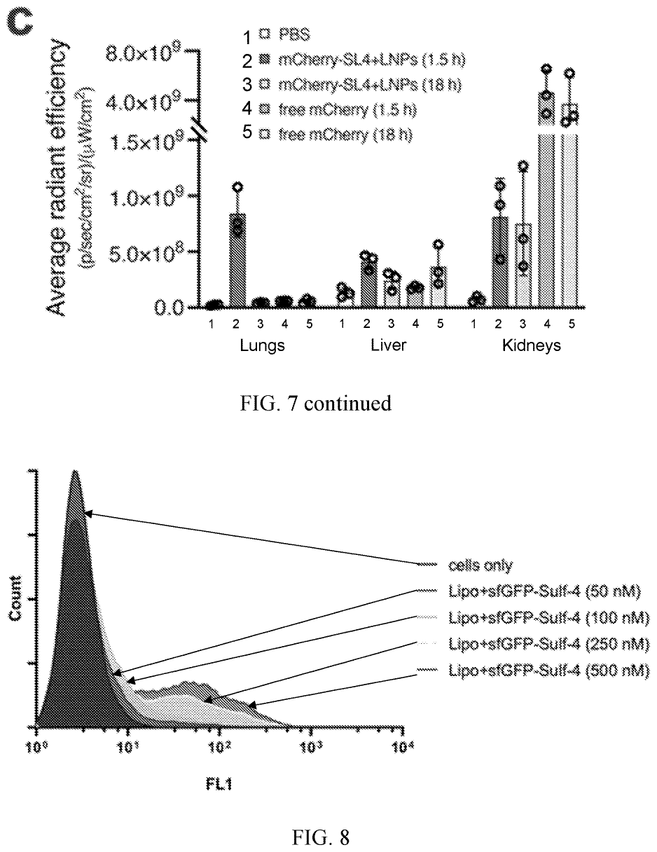

LNP-mediated delivery of cloaked RNase A induces potent cytotoxity. To expand the scope of our delivery platform, we next investigated the functional delivery of ribonuclease A (RNase A), a 13.7-kDa endonuclease that endogenously functions to cleave single-stranded RNAs. High levels of RNase A inside cells can often induce cytotoxic effects,³⁰ which has motivated efforts to promote uptake of RNase A using polymeric- and lipid-based materials³¹⁻³³ for potential anticancer applications. Here, we sought to apply our cloaking strategy to deliver RNase A with LNPs into different cancer cell lines and use cytotoxicity as a phenotypic surrogate to evaluate functional delivery (FIG. 5A).

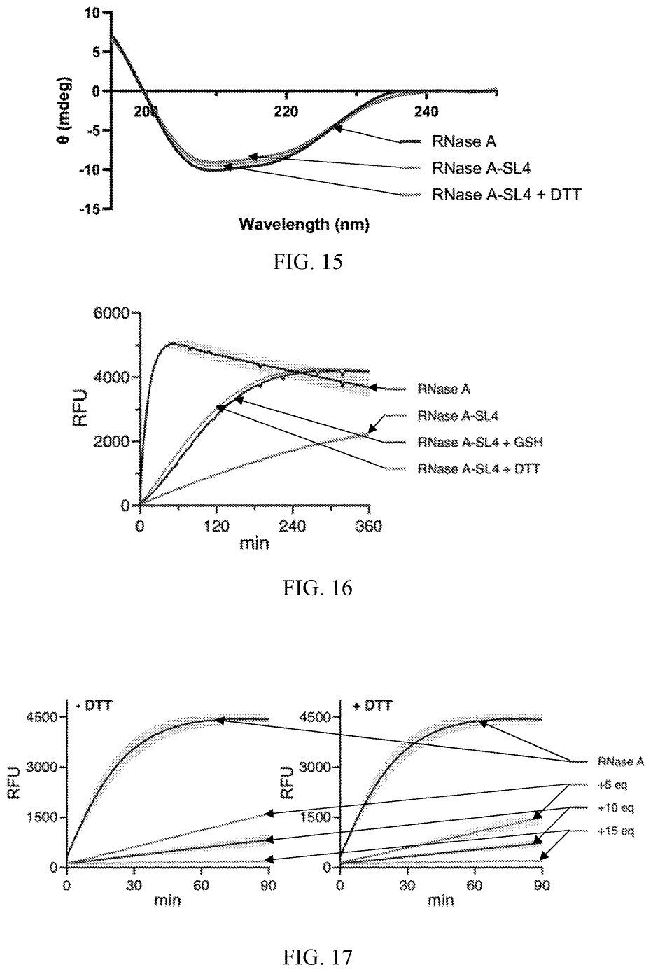

First, we assessed conjugation and anionic modification of RNase A with SL4. RNase A is a highly basic protein (pI≈8.5) and can be readily subjected to efficient charge modification with as little as 5 molar equivalents of SL4, resulting in a pI below 5 (FIG. 13). MALDI-TOF-MS analysis confirmed the attachment of 3 to 5 sulfonated compounds to RNase A modified with 5 to 15 molar equivalents of SL4 (FIG. 14). Circular dichroism (CD) spectra revealed no discernable changes in the secondary structure of RNase A after cloaking with SL4 and after DTT incubation of cloaked RNase A (FIG. 15).

Imperative for functional protein delivery is the ability of a protein cargo to retain its biological activity upon chemical modification. We therefore proceeded to evaluate the activity of RNase A cloaked with SL4 using a standard ribonuclease assay kit. Modification of RNase A with increasing molar equivalents of SL4 reduced nuclease activity, particularly when reacted with 10 or higher molar equivalents of SL4 (FIG. 5B). However, incubation of the cloaked RNase A samples with 10 mM DTT prior to measuring activity resulted in complete recovery of enzymatic activity back to that of the unmodified enzyme, indicating that disulfide cleavage of the sulfonated compounds can restore native protein function. This is further corroborated by recovery of the basic RNase A band in the IEF gel upon incubation with 10 mM GSH, demonstrating successful disulfide cleavage and traceless recovery of the native protein (FIG. 13). Interestingly, co-incubation of cloaked RNase A with 10 mM of either GSH or DTT resulted in gradual recovery of enzyme activity over the course of 6 hours and highlights that cleavage kinetics may play an important role in recovering protein function (FIG. 16).

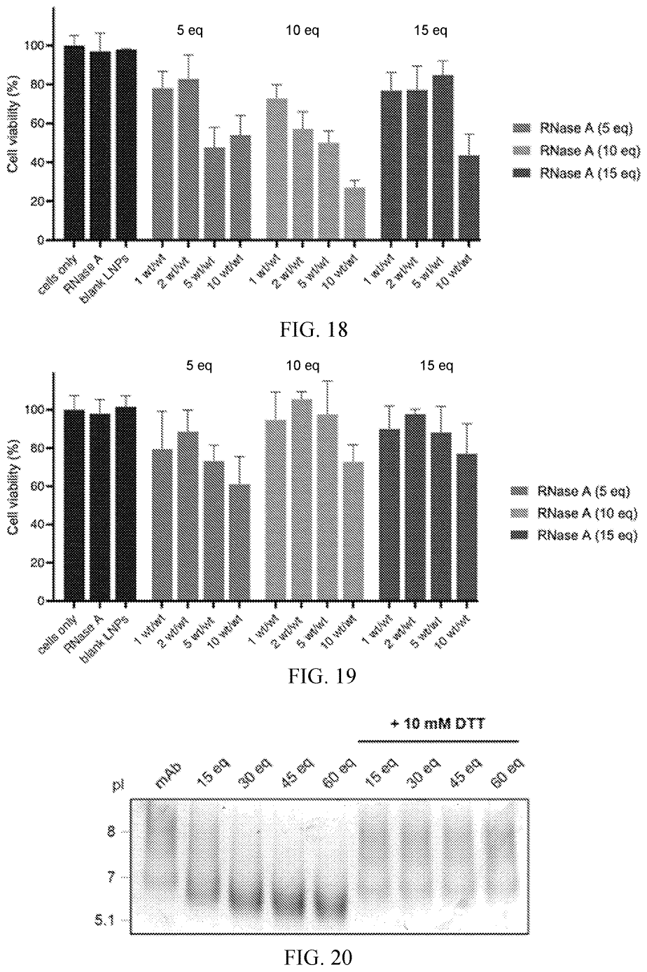

We then delivered anionically-cloaked RNase A into HEK293T cells with various MC3 LNPs (supplemented with 10 mol % DOTAP) to initially optimize formulations for cellular cytotoxicity. Transfections of RNase A cloaked with 5 to 15 molar equivalents of SL4 and formulated into LNPs in pH 5 buffer with varying amounts of lipids (1-10 wt/wt, MC3/RNase A) all resulted in reductions in viability of HEK293T cells (FIG. 18). Maximal reduction in cell viability of nearly 70% was achieved with RNase A-SL4 modified with 10 molar equivalents and formulated with 10 wt/wt, MC3/RNase A (FIG. 5C and FIG. 18). To evaluate the importance of intracellular disulfide linker cleavage for recovery of enzymatic activity, transfections were also performed with RNase A modified with non-redox-cleavable variants of SL4, which led to a less significant reduction in cell viability compared to that of RNase A modified with cleavable compounds (FIG. 5C). The diminished activity of RNase A modified by non-cleavable SL4 was further confirmed from ribonuclease activity assays that demonstrated no recovery in RNase A activity following incubation of cloaked RNase A with 10 mM DTT (FIG. 17).

We next explored cytotoxicity against a wider range of clinically relevant cancer cell lines, including A549 (lung), DLD-1 (colorectal), HeLa (cervical), SK-BR-3 (breast), and SK-OV-3 (ovarian), that vary in size, gene expression profiles, signaling pathways, DNA repair capacity, and cell cycle regulation. Treatment with RNase A modified with 10 molar equivalents of SL4 and formulated with 10 wt/wt, MC3/RNase A resulted in potent dose-dependent reduction in the viability of all tested cancer cells (FIG. 5D), with calculated IC₅₀ values below 400 nM (FIG. 5E). To determine whether the differential responses to RNase A treatment was due to extent of protein delivery or due to unique cancer biology of the tested cell lines, we performed transfections of fluorescein-labeled RNase A-SL4 to quantify the extent of uptake into cells. The half-maximal effective concentration of uptake, EC₅₀, correlated well with the calculated IC₅₀ values for all cancer cells, indicating that the degree of cytotoxicity induced depends on the amount of RNase A delivered (FIG. 5E). Taken together, these results demonstrate that our anionic cloaking method enables efficient delivery of RNase A into a wide variety of cancer cell lines for cancer therapy applications.

Delivery of full-length inhibitory antibodies downregulates β-catenin activity. Immunoglobulin (IgG) antibodies, which possess high affinity and specificity towards their targets, are being increasingly explored for inhibition of intracellular signaling pathways and “undruggable” protein targets. To this end, we explored the feasibility of efficiently delivering an off-the-shelf IgG antibody against β-catenin using our anionic cloaking strategy and LNPs. We selected the transcription factor β-catenin as a target protein for the delivery of inhibitory antibodies because it plays a pivotal role in oncogenic Wnt transduction pathways.³⁶ In Wnt-driven cancer pathogenesis, aberrantly stabilized β-catenin accumulates in the cytosol, translocates to the nucleus, and interacts with TCF/LEF transcription complex to drive the expression of oncogenes including c-Myc and cyclin D1. We hypothesized that anionically-cloaked anti-β-catenin IgG antibodies complexed with MC3 LNPs could be delivered to the cytosol where they would bind stabilized β-catenin and prevent it from entering the nucleus, thereby inhibiting its transcriptional activity (FIG. 6A).

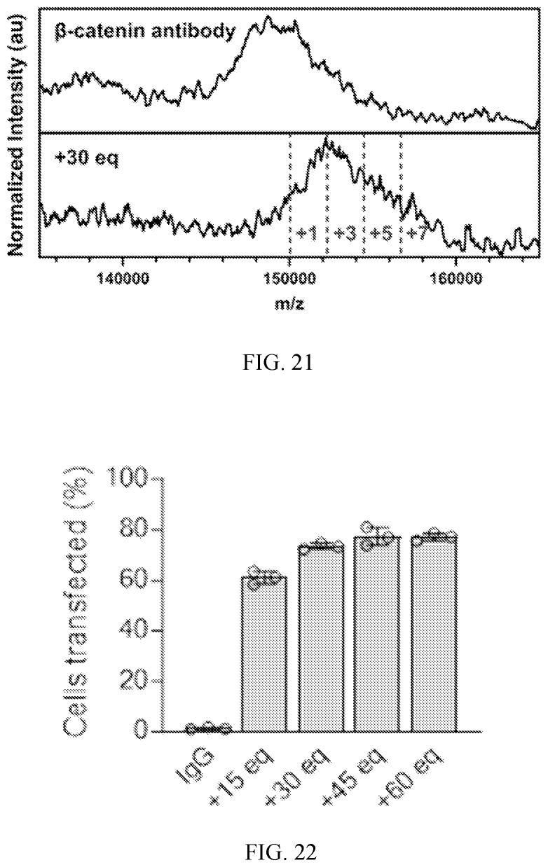

To test this hypothesis, we first investigated cloaking and delivery of a fluorescently labeled mouse anti-rabbit IgG with the goal of optimizing cellular internalization of this large, complex protein cargo. Conjugation with at least 30 molar equivalents of SL4 reduced the pI of the IgG to ˜5 (FIG. 20). Reacting beyond 30 molar equivalents of SL4 had no significant additional impact on pI. Anionically-cloaked IgG was then formulated into LNPs in pH 5 buffer at 2 wt/wt, MC3/antibody and transfected in HEK293T cells. We kept the lipid/protein weight ratio low due to the large molecular weight of the IgG. Flow cytometry analysis revealed that mouse anti-rabbit IgG cloaked with 15-60 molar equivalents of SL4 exhibited 60-80% delivery efficiency into cells (FIG. 21). Notably and consistent with conjugation experiments, delivery efficiency reached a plateau for mouse anti-rabbit IgG modified with over 30 molar equivalents of SL4. It was reasoned that cloaking of IgG with 30 molar equivalents of SL4 was desirable given that protein pI and efficiency of delivery with LNPs do not increase significantly with further modification. Importantly, free IgG antibody in solution and uncloaked IgG antibodies formulated with MC3 LNPs did not internalize into cells (FIG. 6B). It is believed, without intending to be bound to any specific theory, that the anionic cloaking mechanism assists in robust intracellular antibody delivery with LNPs.

We next investigated the delivery of a commercial murine monoclonal antibody specific for β-catenin. Conjugation of this anti-β-catenin IgG was confirmed by MALDI-MS analysis and revealed an average degree of labeling between 3-5 when reacted with 30 molar equivalents of SL4 (FIG. 18 and FIG. 22). The ability of the antibody to bind β-catenin following SL4 conjugation was assessed via quantitative ELISA. While cloaking of the anti-β-catenin IgG with SL4 reduced binding activity to β-catenin, strong binding was largely restored upon incubation of anti-β-catenin IgG-SL4 in the presence of 10 mM DTT (FIG. 6C), suggesting that binding of intracellular β-catenin is possible after cleavage of the anionic cloak following cytosolic delivery.

To test functional β-catenin inhibition, we leveraged the TOPFlash assay, a β-catenin-responsive plasmid reporter comprising TCF binding sites placed upstream of a luciferase expression cassette. As expected, constitutively Wnt-active DLD-1 colorectal cancer cells treated with LNPs only or anti-β-catenin IgG alone exhibited a strong TOPFlash signal (FIG. 6D), indicative of strong β-catenin-mediated transcriptional activity. Excitingly, DLD-1 cells treated with 50-500 nM of cloaked anti-β-catenin IgGs delivered with MC3 LNPs exhibited substantial dose-dependent reduction in TOPFlash signal, whereas DLD-1 cells treated with anionically-cloaked isotype control IgG formulated with MC3 LNPs showed no change in transcriptional activity (FIG. 6D). It should be noted that delivery with as little as 200 nM of anti-β-catenin IgG-SL4 resulted in >60% reduction of β-catenin transcriptional activity. Using a fluoresein-labeled anti-β-catenin IgG-SL4 complexed with MC3 LNPs, we found that update into DLD-1 cells was highly efficient with ˜100% transfection efficiency at 500 nM treatments as determined by flow cytometry (FIG. 19). Nearly identical transfection efficiency was observed for a fluorescein-labeled isotype control IgG-SL4 across the tested concentration ranges, implying that reduction in the TOPFlash signal was a result of specific binding and presumable sequestration of β-catenin following delivery of the anti-β-catenin antibody. Overall, these findings demonstrate the feasibility of employing a readily available, commercial antibody for intracellular cell signaling modulation, thereby paving the way for the repurposing of other off-the-shelf antibodies for various biological and therapeutic applications.

The ability to introduce proteins exogenously into cells presents an immense opportunity to directly manipulate biological functions and translate protein therapies for intracellular applications. In this work, we present a facile bioconjugation strategy for protein delivery with cationic lipids that can be readily applied to virtually any protein cargo. By applying the anionic cloaking strategy on the selected proteins in this study, which vary widely in molecular weight (˜15 kDa to 150 kDa) and surface charge (pI less than 5 and greater than 8), we demonstrate the generalizability of this platform to enable highly efficient delivery into cells using clinically validated LNP formulations.

As is the case for many delivery applications, biological cargos need not only cross cell membranes but also be transported into the cytosol for effecting their intended functions. In this respect, delivery with LNPs, which generally traffic into cells through endosomes, poses a significant challenge towards achieving cytosolic delivery, as it is generally understood that the rate of endosomal escape of LNPs is quite low (less than 5%). The necessity of performing functional delivery experiments, therefore, is crucial for evaluating both cytosolic delivery and biological activity of protein cargos, as access to the cytosol is a requisite step for the downstream functions of most proteins. Our investigations into RNase A and anti-β-catenin IgGs reveal clear indications of successful protein bioactivity following delivery, substantiating compelling evidence of cytosolic delivery. Our studies also demonstrate the importance of redox-mediated disulfide cleavage and self-immolation of the cloaked sites for recovery of protein activity. In cases where protein function is impaired upon anionic cloaking, this provides an exciting opportunity to control kinetics of cleavage (i.e. disulfide linkers that vary in electron-donating/withdrawing pendant groups) for sustained release of protein activity. Additionally, spatial control over protein activity could be achieved through organelle- and tissue-specific linker cleavage mechanisms (i.e. protease-specific cleavable linkers).

An advantage of the anionic cloaking strategy lies in the simplicity of its use—a broad-spectrum reagent that rapidly remodels the surface charge of any protein cargo through the addition of sulfonate groups, a chemical group that is not present in the toolkit of canonical amino acids. This versatile delivery platform holds the potential to repurpose a wide range of commercial and therapeutic proteins for novel intracellular applications.

Although the present disclosure has been described with respect to one or more particular embodiments and/or examples, it will be understood that other embodiments and/or examples of the present disclosure may be made without departing from the scope of the present disclosure.

1. An anionically functionalized polypeptide comprising the following structure: D-G-X₁-L-(R)ₓ, wherein
D comprises a peptide group;
G comprises a conjugated group;
X₁ comprises a cleavable group;
L comprises a linking group;
R comprises an anionic group; and
x is 1, 2, 3, 4, 5, or 6.

2. The anionically functionalized polypeptide of claim 1, wherein the polypeptide comprises the following structure: 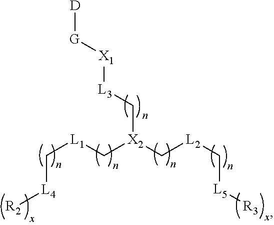
wherein
X₂ is chosen from an N group, a CH group, a P group, and a P═O group;
L₁ and L₂ are independently chosen from —OC(O)—, —C(O)O—, —NHC(O)—, —C(O)NH—, —OC(O)NH—, —NHC(O)O—, 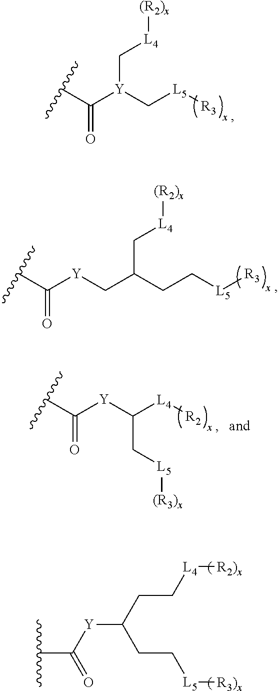
wherein Y is independently chosen from a NH group, an O group, an S group, a CH₂ group, an H group, and a CH group;
L₃ is chosen from —OC(O)—, —C(O)O—, —NHC(O)—, —C(O)NH—, —OC(O)NH—, —NHC(O)O—;
L₄ and L₅ are independently optional and chosen from alkyl groups and alkenyl groups;
R₂ and R₃ are independently optional, wherein the anionically functionalized polypeptide comprises at least one R₂ and/or R₃, and chosen from —CO₂⁻, —CO₂H, —SO₃⁻, —SO₃H, —SO₂⁻, —PO₂H²⁻, —PO₃H⁻, and —PO₄H²⁻;
n is independently at each occurrence 0, 1, 2, 3, 4, 5, or 6; and
x is independently at each occurrence 1, 2, 3, 4, 5, or 6.

3. The anionically functionalized polypeptide of claim 2, wherein the polypeptide comprises the following structure:

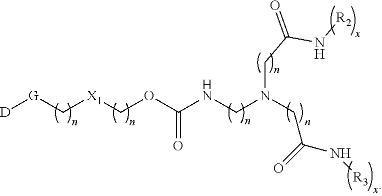

4. The anionically functionalized polypeptide of claim 3, wherein the polypeptide comprises the following structure:

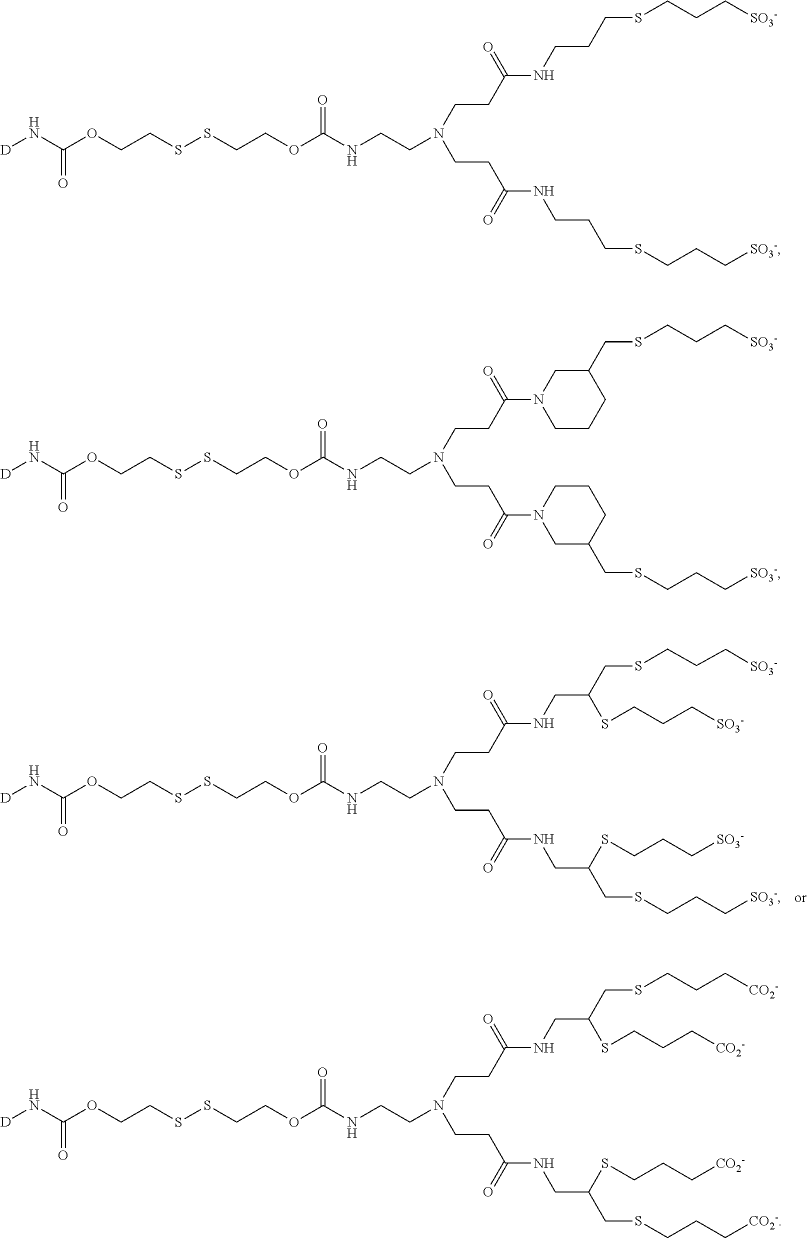

5. The anionically functionalized polypeptide of claim 1, wherein the peptide group is chosen from enzyme groups, receptor ligand, transcriptional factor groups, growth factor groups, antibody groups, peptide or protein immunogens, protein-based therapeutic agent groups, toxin groups, cytokine groups, hormone groups, fluorescent protein groups, any fragments thereof, any modified structural analogs thereof, and any combination thereof.

6. The anionically functionalized polypeptide of claim 5, wherein the enzyme groups are chosen from Clustered Regularly Interspaced Short Palindromic Repeats (CRISPR) enzyme groups.

7. The anionically functionalized polypeptide of claim 1, wherein the conjugated group comprises the following structure: 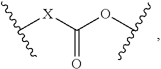
wherein X is chosen from an N group, an S group, and an O group.

8. The anionically functionalized polypeptide of claim 1, wherein the cleavable group is chosen from stimuli-cleavable bonds, light-cleavable bonds, ROS-cleavable bonds, and pH-cleavable bonds.

9. The anionically functionalized polypeptide of claim 8, wherein the stimuli-cleavable bond comprises a disulfide bond.

10. A composition comprising one or more anionically functionalized polypeptide(s) of claim 1.

11. The composition of claim 10, wherein the composition further comprises one or more cationic lipid(s) or a combination of lipids.

12. The composition of claim 10, wherein the composition comprises a plurality of lipoplexes or a plurality of lipid nanoparticles, wherein the anionically functionalized polypeptide(s) is/are independently disposed in or partially or completely encapsulated by a lipid nanoparticle.

13. The composition of claim 10, wherein the composition is a pharmaceutical composition, and the composition further comprises one or more pharmaceutically acceptable excipient(s).

14. A method of treating a subject with one or more symptoms(s), the method comprising:
administering to a subject an effective amount of one or more anionically functionalized polypeptide(s) of claim 1,
wherein at least one of the one or more symptom(s) of the subject is at least partially alleviated.

15. A bioconjugation reagent comprising the following structure: G′-X₁-L-(R)ₓ, wherein
G′ comprises a conjugation group;
X₁ comprises a cleavable group;
L comprises a linking group;
R comprises an anionic group; and
x is 1, 2, 3, 4, 5, or 6.

16. The bioconjugation reagent of claim 15, wherein the bioconjugation reagent comprises the following structure: 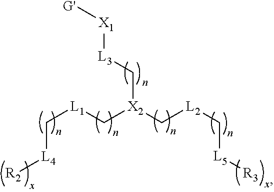
wherein
X₂ is chosen from an N group, a CH group, a P group, and a P═O group;
L₁ and L₂ are independently chosen from —OC(O)—, —C(O)O—, —NHC(O)—, —C(O)NH—, —OC(O)NH—, —NHC(O)O—, 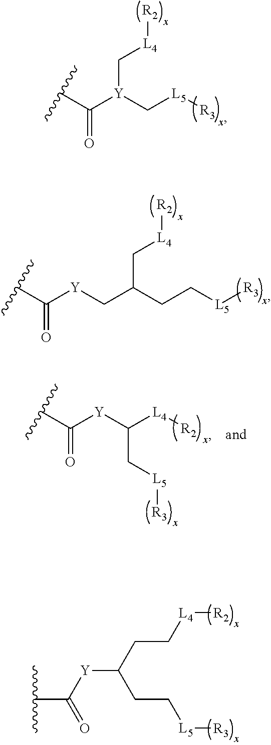
wherein Y is independently chosen from an NH group, an O group, an S group, a CH₂ group, an H group, and a CH group;
L₃ is chosen from —OC(O)—, —C(O)O—, —NHC(O)—, —C(O)NH—, —OC(O)NH—, —NHC(O)O—;
L₄ and L₅ are independently optional and chosen from an alkyl group and an alkenyl group;
R₂ and R₃ are independently optional, wherein the bioconjugation reagent comprises at least one R₂ and/or R₃, and chosen from —CO₂⁻, —CO₂H, —SO₃⁻, —SO₃H, —SO₂⁻, —PO₂H²⁻, —PO₃H⁻, and —PO₄H²⁻;
n is independently at each occurrence 0, 1, 2, 3, 4, 5, or 6; and
x is independently at each occurrence 1, 2, 3, 4, 5, or 6.

17. The bioconjugation reagent of claim 16, wherein the bioconjugation reagent comprises the following structure:

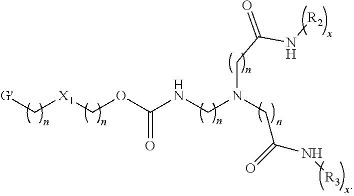

18. (canceled)

19. The bioconjugation reagent of claim 15, wherein the conjugation group is chosen from carbonate groups and ester groups and/or the cleavable group comprises a stimuli-cleavable bond chosen from redox-cleavable disulfide bonds, light-cleavable bonds, ROS-cleavable bonds, and pH-cleavable bonds.

20. (canceled)

21. (canceled)

22. (canceled)

23. A method of making one or more anionically functionalized polypeptide(s) of claim 1, comprising:
forming a reaction mixture comprising
one or more bioconjugation reagent(s) of claim 14, and
one or more polypeptide(s); and,
holding the reaction mixture for a time and/or temperature,
wherein the anionically functionalized polypeptide(s) is/are formed.

24. (canceled)

25. (canceled)

26. (canceled)

27. The method of claim 23, further comprising modifying at least a portion or all of the anionically functionalized polypeptide(s).

28. (canceled)

29. (canceled)


SEQUENCE LISTING (17 sequences)

SEQ ID NO: 1 - protein, 7 aa, synthetic construct
PKKKRKV

SEQ ID NO: 2 - protein, 10 aa, synthetic construct
PKKKRKVEAS

SEQ ID NO: 3 - protein, 16 aa, synthetic construct
KRPAATKKAGQAKKKK

SEQ ID NO: 4 - protein, 9 aa, synthetic construct
PAAKRVKLD

SEQ ID NO: 5 - protein, 11 aa, synthetic construct
RQRRNELKRSP

SEQ ID NO: 6 - protein, 38 aa, synthetic construct
NQSSNFGPMKGGNFGGRSSGPYGGGGQYFAKPRNQGGY

SEQ ID NO: 7 - protein, 42 aa, synthetic construct
RMRIZFKNKGKDTAELRRRRVEVSVELRKAKKDEQILKRRNV

SEQ ID NO: 8 - protein, 8 aa, synthetic construct
VSRKRPRP

SEQ ID NO: 9 - protein, 8 aa, synthetic construct
PPKKARED

SEQ ID NO: 10 - protein, 8 aa, synthetic construct
PQPKKKPL

SEQ ID NO: 11 - protein, 12 aa, synthetic construct
SALIKKKKKMAP

SEQ ID NO: 12 - protein, 5 aa, synthetic construct
DRLRR

SEQ ID NO: 13 - protein, 7 aa, synthetic construct
PKQKKRK

SEQ ID NO: 14 - protein, 10 aa, synthetic construct
RKLKKKIKKL

SEQ ID NO: 15 - protein, 10 aa, synthetic construct
REKKKFLKRR

SEQ ID NO: 16 - protein, 20 aa, synthetic construct
KRKGDEVDGVDEVAKKKSKK

SEQ ID NO: 17 - protein, 17 aa, synthetic construct
RKCLQAGMNLEARKTKK